(19) United States
(12) Patent Application Publication
(10) Pub. No.: US 2023/0028059 A1
(43) Pub. Date: Jan. 26, 2023

(54) MULTI-LEVEL SURGICAL DATA ANALYSIS SYSTEM

(71) Applicant: Cilag GmbH International, Zug (CH)

(72) Inventors: Frederick E. Shelton, IV, Hillsboro, OH (US); Matjaz Jogan, Philadelphia, PA (US); Jason L. Harris, Lebanon, OH (US)

(21) Appl. No.: 17/384,151

(22) Filed: Jul. 23, 2021

Related U.S. Application Data
(60) Provisional application No. 63/224,813, filed on Jul. 22, 2021.

(51) Int. Cl. G16H 30/40; A61B 17/00; G06N 20/00; A61B 34/30

(52) U.S. Cl. CPC G16H 30/40; A61B 17/00; G06N 20/00; A61B 34/30; A61B 2017/00221

(57) ABSTRACT

A computing system may obtain, from surgical hub(s) and/or other system(s), collections of unredacted data associated with different surgical procedures. The computing system, the surgical hub(s), and other systems may be located on a local data network. The local data network may be within a boundary protected by health insurance portability and accountability act (HIPAA) data rules. The computing system may train machine learning model(s) based on the unredacted data. The computing system may generate information that optimizes the clinical outcome and cost effectiveness of future surgical procedure(s) based on the machine learning model(s). The computing system may send generated information to the surgical hub(s) and/or other system(s). The computing system may be in communication with a remote cloud computing system. The computing system may send the generated information to the remote cloud computing system.

### CROSS-REFERENCE TO RELATED APPLICATIONS

This application claims the benefit of Provisional U.S. Patent Application No. 63/224,813, filed Jul. 22, 2021, the disclosure of which is incorporated herein by reference in its entirety.

### BACKGROUND

Various systems may operate at medical facilities (e.g., hospitals). The systems may exchange various types of data with each other. Such data may be protected by privacy rules mandated by authorities. Such data may be analyzed to generate various types of analytics. For example, the systems may exchange data associated with surgical procedures with each other. The data associated with surgical procedures may be protected by health insurance portability and accountability act (HIPAA) rules. The data associated with surgical procedures may be analyzed to generate analytics.

### SUMMARY

Systems, methods, and instrumentalities are described herein for surgical data analysis. A computing system may obtain, from surgical hub(s) and/or other system(s), collections of unredacted data associated with different surgical procedures. The computing system, the surgical hub(s), and other system(s) may be located on a local data network. The local data network may be within a boundary protected by health insurance portability and accountability act (HIPAA) data rules.

The computing system may train machine learning model(s) based on the unredacted data. The computing system may generate information that optimizes the clinical outcome and cost effectiveness of future surgical procedure(s) based on the machine learning model(s). The computing system may send generated information to the surgical hub(s) and/or other system(s). The computing system may be in communication with a remote cloud computing system. The computing system may send the generated information to the remote cloud computing system.

### BRIEF DESCRIPTION OF THE DRAWINGS

FIG. 1A is a block diagram of a computer-implemented surgical system.

FIG. 1B is a block diagram of a computer-implemented multi-tier surgical system.

FIG. 1C is a logical diagram illustrating control plane and data plane of a surgical system.

FIG. 2 shows an example surgical system in a surgical operating room.

FIG. 3 illustrates an example surgical hub paired with various systems.

FIG. 4 illustrates a surgical data network having a set of communication surgical hubs configured to connect with a set of sensing systems, an environmental sensing system, a set of devices, etc.

FIG. 5 illustrates an example computer-implemented interactive surgical system that may be part of a surgical system.

FIG. 6 illustrates a logic diagram of a control system of a surgical instrument.

FIG. 7 shows an example surgical system that includes a handle having a controller and a motor, an adapter releasably coupled to the handle, and a loading unit releasably coupled to the adapter.

FIG. 8 shows an example situationally aware surgical system.

FIG. 9 shows an example multi-level surgical data analysis system.

FIG. 10 is a block diagram of an example edge computing system operating with an intra-surgical hub network.

FIG. 11 is the flow chart of an example operation of an edge computing system.

### DETAILED DESCRIPTION

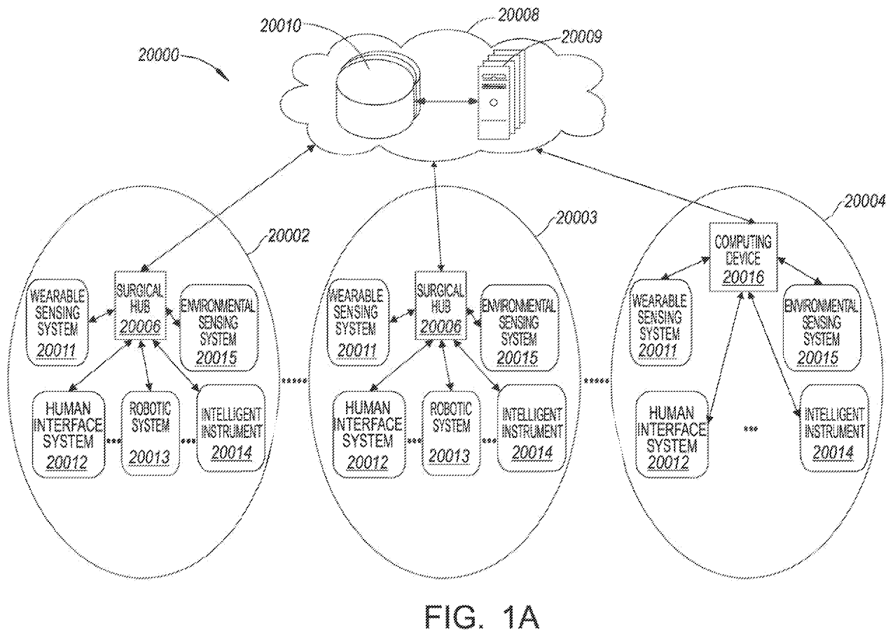

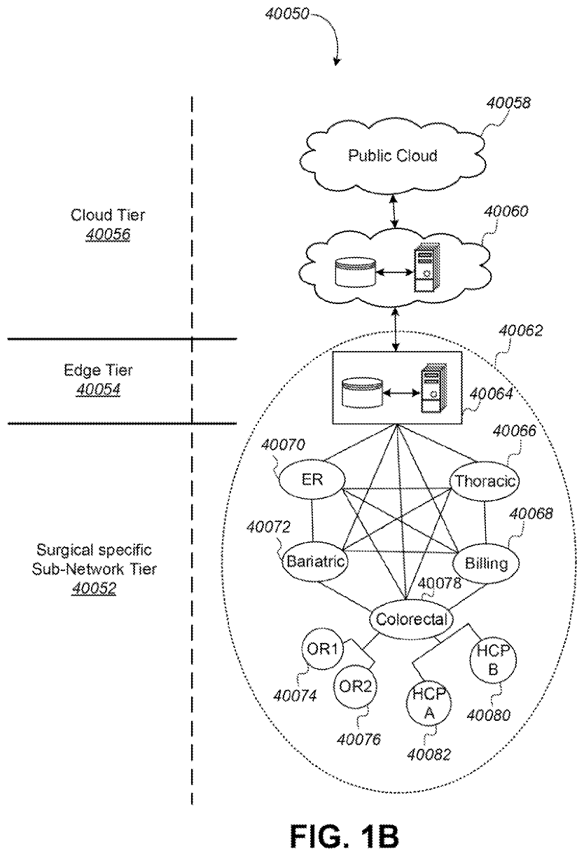

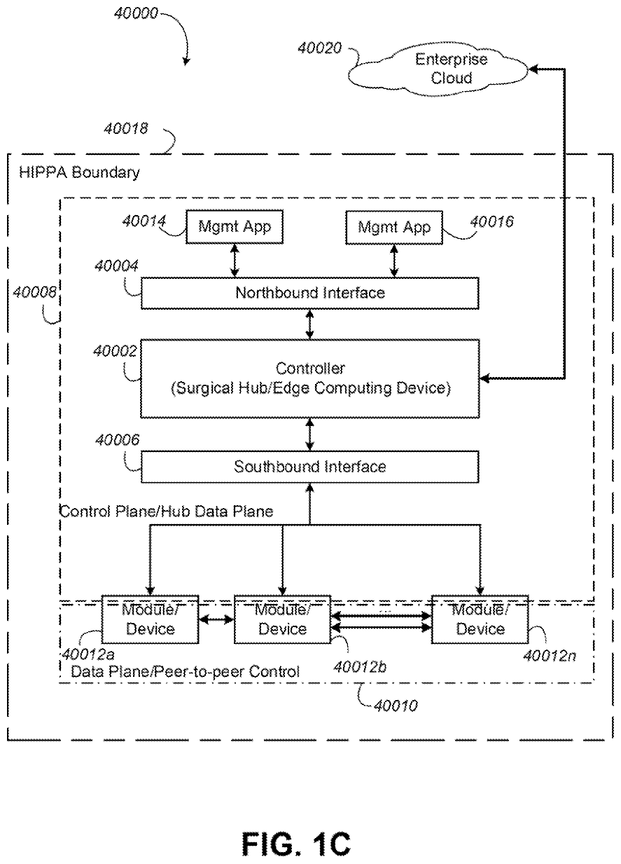

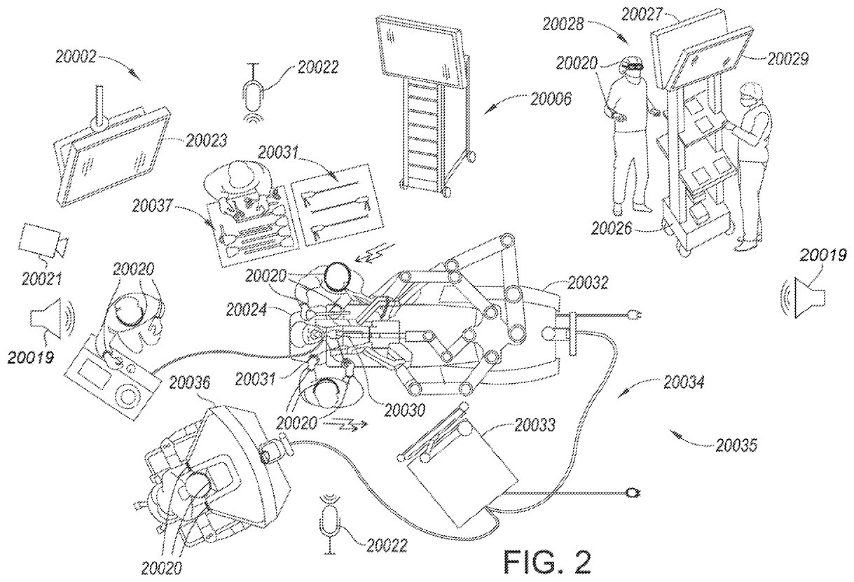

FIG. 1A is a block diagram of a computer-implemented surgical system 20000. An example surgical system such as the surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include a wearable sensing system 20011, an environmental sensing system 20015, a robotic system 20013, one or more intelligent instruments 20014, human interface system 20012, etc. The human interface system is also referred herein as the human interface device. The wearable sensing system 20011 may include one or more HCP sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G.

A surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the one or more sensing systems 20011 and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems 20001 may include the wearable sensing system 20011 (which may include one or more HCP sensing systems and one or more patient sensing systems) and the environmental sensing system 20015 as discussed in FIG. 1A. The one or more sensing systems 20001 may measure data relating to various biomarkers. The one or more sensing systems 20001 may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The one or more sensors may measure the biomarkers as described herein using one of more of the following sensing technologies: photoplethysmography, electrocardiography, electroencephalography, colorimetry, impedimentary, potentiometry, amperometry, etc.

The biomarkers measured by the one or more sensing systems 20001 may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example. The one or more sensing systems 20001, biomarkers 20005, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

FIG. 1B is a block diagram of a computer-implemented multi-tier surgical system. As illustrated in FIG. 1B, a computer-implemented multi-tier surgical system 40050 may include multiple tiers of systems, such as a surgical specific sub-network tier system 40052, an edge tier system 40054 that is associated with the surgical specific sub-network tier system 40052, and a cloud tier system 40056.

A surgical specific sub-network tier system 40052 may include a plurality of interconnected surgical sub-systems. For example, the surgical sub-systems may be grouped by the type of surgical procedures and/or other departments in a medical facility or a hospital. For example, a medical facility or a hospital may include a plurality of surgical procedure specific departments, such as an emergency room (ER) department 40070, colorectal department 40078, bariatric department 40072, thoracic department 40066, and billing department 40068. Each of the surgical procedure specific departments may include one or more surgical sub-systems associated with an operating room (OR) and/or a healthcare care professional (HCP). For example, the colorectal department 40078 may include a set of surgical hubs (e.g., surgical hub 20006 as described in FIG. 1A). The surgical hubs may be designated for a respective HCP, such as HCP A, 40082 and HCP B, 40080. In an example, the colorectal department may include a group of surgical hubs that may be located in respective ORs, such as OR 1, 40074 and OR 2, 40076. The medical facility or the hospital may also include a billing department subsystem 40068. The billing department subsystem 40068 may store and/or manage billing data associated with a respective department, such as the ER department 40070, colorectal department 40078, bariatric department 40072, and/or thoracic department 40066.

An edge tier system 40054 may be associated with a medical facility or a hospital and may include one or more edge computing systems 40064, for example. An edge computing system 40064 may include a storage sub-system and a server sub-system. In an example, the edge computing system comprising an edge server and/or a storage unit may provide additional processing and/or storage services to a surgical hub that is part of one of the departmental ORs (e.g., OR1 and OR2 of the colorectal department).

The surgical specific sub-network tier system 40052 and the edge tier system 40054 may be located within a Health Insurance Portability and Accountability Act (HIPAA) boundary 40062. The surgical specific sub-network system 40052 and the edge tier system 40054 may be connected to the same local data network. The local data network may be a local data network of a medical facility or a hospital. The local data network may be within the HIPAA boundary. Because the surgical specific sub-network tier system 40052 and the edge tier system 40054 are located within the HIPAA boundary 40062, patient data between an edge computing system 40064 and a device located within one of the entities of the surgical specific sub-network tier system 40052 may flow without redaction and/or encryption. For example, patient data between an edge computing system 40064 and a surgical hub located in OR1 40074 of the colorectal department 40078 may flow without redaction and/or encryption.

The cloud tier system 40056 may include an enterprise cloud system 40060 and a public cloud system 40058. For example, the enterprise cloud system 40060 may be a cloud computing system 20008 that includes a remote cloud server sub-system and/or a remote cloud storage subsystem, as described in FIG. 1A. The enterprise cloud system 40060 may be managed by an organization, such as a private company. The enterprise cloud system 40060 may be in communication with one or more entities (e.g., edge computing systems 40064, surgical hubs located in ORs (e.g., OR1 40074) of the various departments (e.g., colorectal department 40078)) that are located within the HIPAA boundary 40062.

The public cloud system 40058 may be operated by a cloud computing service provider. For example, the cloud computing service provider may provide storage services and/or computing services to a plurality of enterprise cloud systems (e.g., enterprise cloud system 40060).

FIG. 1C is a logical block diagram 40000 illustrating various communication planes in a surgical system. As illustrated in FIG. 1C, the communication planes between a controller 40002 and management applications 40014 and 40016 on one side and, the system modules and/or modular devices 40012a through 40012n on the other side, may use control plane 40008 and data plane 40010. In an example, in addition to the control plane 40008, a data plane may also exist between the system modules and/or modular devices 40012a through 40012n and the surgical hub. The data plane 40010 may provide data plane paths (e.g., redundant data plane paths) between the system modules and/or the modular devices 40012a through 40012n that are associated with one or more surgical hubs. A surgical hub or one of the surgical hubs (e.g., in case of a plurality of surgical hubs present in an operating room) may act as a controller 40002. In an example, the controller 40002 may be an edge computing system that may reside within a Health Insurance Portability and Accountability Act (HIPAA) boundary where the surgical system is located, for example, as illustrated in FIG. 1B. The controller 40002 may be in communication with an enterprise cloud system 40020. As illustrated in FIG. 1C, the enterprise cloud system 40020 may be located outside the HIPAA boundary 40018. Accordingly, the patient data flowing to and/or from the enterprise cloud system 40020 may be redacted and/or encrypted.

The controller 40002 may be configured to provide a northbound interface 40004 and a southbound interface 40006. The northbound interface 40004 may be used for providing a control plane 40008. The control plane 40008 may include one or more management applications 40014 and 40016 that may enable a user to configure and/or manage system modules and/or modular devices modular devices 40012a through 40012n associated with a surgical system. The management applications 40014 and 40016 may be used to obtain status of various system modules and/or the modular devices 40012a through 40012n.

The management applications 40014 and 40016 using the control plane may interact with the controller 40002, for example, using a set of application programming interface (API) calls. The management applications 40014 and 40016 may interact with the controller 40002 via a management protocol or an application layer protocol to configure and/or monitor the status of a system module and/or a modular device. The management protocols or the application layer protocols used to monitor the status and/or configure a system module or a modular device associated with a surgical system may include the simple network management protocol (SNMP), TELNET protocol, secure shell (SSH) protocol, network configuration protocol (NETCONF), etc.

SNMP or a similar protocol may be used to collect status information and/or send configuration related data (e.g., configuration related control programs) associated with system modules and/or modular devices to the controller. SNMP or a similar protocol may collect information by selecting devices associated with a surgical system from a central network management console using messages (e.g., SNMP messages). The messages may be sent and/or received at fixed or random intervals. The messages may include Get messages and Set messages. The Get messages or messages similar to the Get messages may be used for obtaining information from a system module or a modular device associated with a surgical system. The Set message or messages similar to the Set message may be used for changing a configuration associated with a system module or a modular device associated with a surgical system.

For example, the Get messages or similar messages may include the SNMP messages GetRequest, GetNextRequest, or GetBulkRequest. The Set messages may include SNMP SetRequest message. The GetRequest, GetNextRequest, GetBulkRequest messages or similar messages may be used by a configuration manager (e.g., an SNMP manager) running on the controller 40002. The configuration manager may be in communication with a communication agent (e.g., an SNMP agent) that may be a part of a system module and/or a modular device in a surgical system. The SNMP message SetRequest message or similar may be used by the communication manager on the controller 40002 to set the value of a parameter or an object instance in the communication agent on a system module and/or a modular device of a surgical system. In an example, SNMP modules, for example, may be used to establish communication path between system modules and/or modular devices associated with a surgical system.

Based on the query or configuration related messages received from a management application, such as management applications 40014 and 40016, the controller 40002 may generate configuration queries and/or configuration data for querying or configuring the system modules and/or the modular devices associated with the surgical hub or the surgical system. A surgical hub (e.g., the surgical hub 20006 shown in FIG. 1A) or an edge computing system (e.g., the edge computing system 40064 shown in FIG. 1B) may manage and/or control various system modules and/or modular devices 40012a through 40012n associated with a surgical system. For example, the northbound interface 40004 of the controller 40002 may be used for changing control interactions between one or more modules associated and/or devices associated with a surgical system. In an example, the controller 40002 may be used for establishing one or more communication data paths between a plurality of modules and/or devices associated with a surgical system. The controller 40002 may use its southbound interface 40006 to send the control programs comprising queries and/or configuration changes to the system modules and/or the modular devices of the surgical system.

The system modules and/or the modular devices 40012a through 40012n of a surgical system, or the communication agents that may be a part of the system modules and/or the modular devices, may send notification messages or traps to the controller 40002. The controller may forward the notification messages or traps via its northbound interface 40004 to the management application 40014 and 40016 for displaying on a display. In an example, the controller 40002 may send the notification to other system modules and/or modular devices 40012a through 40012n that are part of the surgical system.

The system modules and/or the modular devices 40012a through 40012n of a surgical system or the communication agents that are part of the system modules and/or the modular devices may send responses to the queries received from the controller 40002. For example, a communication agent that may be part of a system module or a modular device may send a response message in response to a Get or a Set message or messages similar to the Get or the Set messages received from the controller 40002. In an example, in response to a Get message or a similar message received from the controller 40002, the response message from the system module or the modular device 40012a through 40012n may include the data requested. In an example, in response to a Set message or a similar message received from a system module or a modular device 40012a through 40012n, the response message from the controller 40002 may include the newly set value as confirmation that the value has been set.

A trap or a notification message or a message similar to the trap or the notification message may be used by a system module or a modular device 40012a through 40012n to provide information about events associated with the system modules or the modular devices. For example, a trap or a notification message may be sent from a system module or a modular device 40012a through 40012n to the controller 40002 indicating a status of a communication interface (e.g., whether it available or unavailable for communication). The controller 40002 may send a receipt of the trap message back to the system module or the modular device 40012a through 40012n (e.g., to the agent on the system module or a modular device).

In an example, TELNET protocol may be used to provide a bidirectional interactive text-oriented communication facility between system modules and/or modular devices 40012a through 40012n and the controller 40002 TELNET protocol may be used to collect status information and/or send configuration data (e.g., control programs) from/to the controller 40002. TELNET may be used by one of the management applications 40014 or 40016 to establish a connection with the controller 40002 using the transmission control protocol port number 23.

In an example, SSH, a cryptographic encrypted protocol, may be used to allow remote login and to collect status information and/or send configuration data about system modules and/or modular devices 40012a through 40012n from/to the controller 40002. SSH may be used by one of the management applications 40014 or 40016 to establish an encrypted connection with the controller 40002 using the transmission control protocol port number 22.

In an example, NETCONF may be used to perform management functions by invoking remote procedure calls using, for example, <rpc>, <rpc-reply>, or <edit-config> operations. The <rpc> and <rpc-reply> procedure calls or similar procedure calls may be used for exchanging information from a system module and/or a modular device associated with a surgical system. The NETCONF <edit-config> operation or a similar operation may be used for configuring the system modules and/or the modular devices associated with the surgical system.

The controller 40002 may configure the system modules and/or modular device 40012a through 40012n to establish a data plane 40010. The data plane 40010 (e.g., also referred to as a user plane or a forwarding plane) may enable a communication data path between a plurality of system modules and/or modular device 40012a through 40012n. The data plane 40010 may be utilized by the system modules and/or the modular device 40012a through 40012n for communicating data flows of data between the system modules and/or modular devices associated with a surgical system. The data flows may be established using one or more dedicated communication interfaces between the system modules and/or the modular devices associated with one or more surgical hubs of a surgical system. In an example, the data flows may be established over one or more local area networks (LANs) and one or more wide area networks (WANs), such as the Internet.

In an example, the data plane 40010 may provide support for establishing a first and a second independent, disjointed, concurrent, and redundant communication path for data flow between the system modules and/or modular devices 40012b and 40012n. As illustrated in FIG. 1C. redundant communication paths may be established between system modules/modular devices 40012b and 40012n. The redundant communication paths may carry same/redundant data flows between the system modules and/or modular devices. In an example, when or if some of the data packets are dropped on one of the redundant communication paths due to problems with one of the communication interfaces on the system modules/modular devices 40012b and 40012n, the system modules and/or the modular devices may continue to send/receive at least one copy of the dropped data packets over the second communication path.

FIG. 2 shows an example of a surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1A. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

In one aspect, the surgical hub 20006 may be configured to route a diagnostic input or feedback entered by a non-sterile operator at the visualization tower 20026 to the primary display 20023 within the sterile field, where it can be viewed by a sterile operator at the operating table. In one example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

FIG. 2 illustrates an example of a surgical system 20002 being used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (i.e., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1A may include one or more sensing systems, for example, HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, a sensing system 20020 may measure a set of biomarkers to monitor the heart rate of an HCP. In an example, a sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing. One or more environmental sensing devices may send environmental information to the surgical hub 20006. For example, the environmental sensing devices may include a camera 20021 for detecting hand/body position of an HCP.

The environmental sensing devices may include microphones 20022 for measuring the ambient noise in the surgical theater. Other environmental sensing devices may include devices, for example, a thermometer to measure temperature and a hygrometer to measure humidity of the surroundings in the surgical theater, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors. In an example, the HCP sensing systems 20020 may measure one or more surgeon biomarkers associated with an HCP and send the measurement data associated with the surgeon biomarkers to the surgical hub 20006. The HCP sensing systems 20020 may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

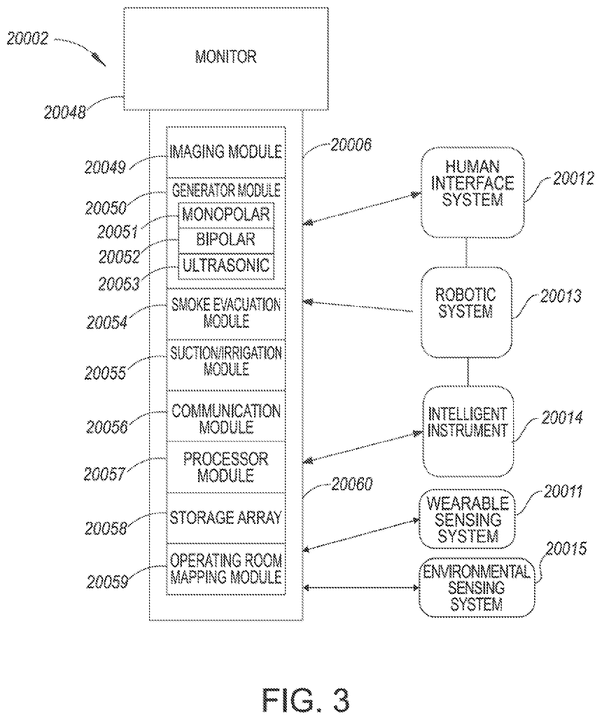

FIG. 3 shows an example surgical system 20002 with a surgical hub 20006 paired with a wearable sensing system 20011, an environmental sensing system 20015, a human interface system 20012, a robotic system 20013, and an intelligent instrument 20014. The hub 20006 includes a display 20048, an imaging module 20049, a generator module 20050, a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure 20060 offers a unified environment for managing the power, data, and fluid lines, which reduces the frequency of entanglement between such lines. Aspects of the present disclosure present a surgical hub 20006 for use in a surgical procedure that involves energy application to tissue at a surgical site. The surgical hub 20006 includes a hub enclosure 20060 and a combo generator module slidably receivable in a docking station of the hub enclosure 20060. The docking station includes data and power contacts. The combo generator module includes two or more of an ultrasonic energy generator component, a bipolar RF energy generator component, and a monopolar RF energy generator component that are housed in a single unit. In one aspect, the combo generator module also includes a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. In one aspect, the fluid line may be a first fluid line, and a second fluid line may extend from the remote surgical site to a suction and irrigation module 20055 slidably received in the hub enclosure 20060. In one aspect, the hub enclosure 20060 may include a fluid interface. Certain surgical procedures may require the application of more than one energy type to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. One of the advantages of the hub modular enclosure 20060 is enabling the quick removal and/or replacement of various modules. Aspects of the present disclosure present a modular surgical enclosure for use in a surgical procedure that involves energy application to tissue. The modular surgical enclosure includes a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. Further to the above, the modular surgical enclosure also includes a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module. Referring to FIG. 3, aspects of the present disclosure are presented for a hub modular enclosure 20060 that allows the modular integration of a generator module 20050, a smoke evacuation module 20054, and a suction/irrigation module 20055. The hub modular enclosure 20060 further facilitates interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 can be configured to connect to a monopolar device 20051, a bipolar device 20052, and an ultrasonic device 20053. Alternatively, the generator module 20050 may comprise a series of monopolar, bipolar, and/or ultrasonic generator modules that interact through the hub modular enclosure 20060. The hub modular enclosure 20060 can be configured to facilitate the insertion of multiple generators and interactive communication between the generators docked into the hub modular enclosure 20060 so that the generators would act as a single generator.

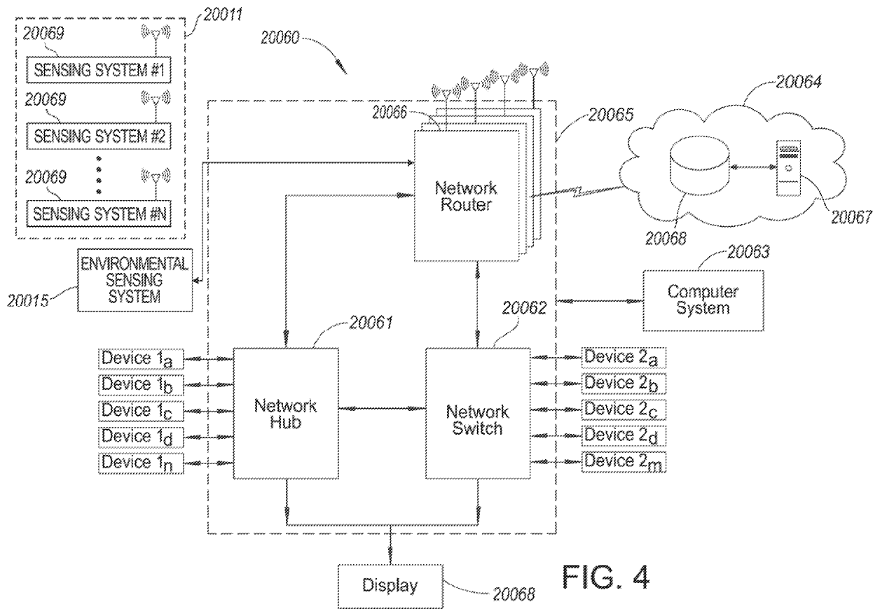

FIG. 4 illustrates a surgical data network having a set of communication hubs configured to connect a set of sensing systems, environment sensing system(s), and a set of other modular devices located in one or more operating theaters of a healthcare facility, a patient recovery room, or a room in a healthcare facility specially equipped for surgical operations, to the cloud, in accordance with at least one aspect of the present disclosure.

As illustrated in FIG. 4, a surgical hub system 20060 may include a modular communication hub 20065 that is configured to connect modular devices located in a healthcare facility to a cloud-based system (e.g., a cloud computing system 20064 that may include a remote server 20067 coupled to a remote storage 20068). The modular communication hub 20065 and the devices may be connected in a room in a healthcare facility specially equipped for surgical operations. In one aspect, the modular communication hub 20065 may include a network hub 20061 and/or a network switch 20062 in communication with a network router 20066. The modular communication hub 20065 may be coupled to a local computer system 20063 to provide local computer processing and data manipulation.

The computer system 20063 may comprise a processor and a network interface 20100. The processor may be coupled to a communication module, storage, memory, non-volatile memory, and input/output (I/O) interface via a system bus. The system bus can be any of several types of bus structure(s) including the memory bus or memory controller, a peripheral bus or external bus, and/or a local bus using any variety of available bus architectures including, but not limited to, 9-bit bus, Industrial Standard Architecture (ISA), Micro-Charmel Architecture (MSA), Extended ISA (EISA), Intelligent Drive Electronics (IDE), VESA Local Bus (VLB), Peripheral Component Interconnect (PCI), USB, Advanced Graphics Port (AGP), Personal Computer Memory Card International Association bus (PCMCIA), Small Computer Systems Interface (SCSI), or any other proprietary bus.

The processor may be any single-core or multicore processor such as those known under the trade name ARM Cortex by Texas Instruments. In one aspect, the processor may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle serial random access memory (SRAM), an internal read-only memory (ROM) loaded with StellarisWare® software, a 2 KB electrically erasable programmable read-only memory (EEPROM), and/or one or more pulse width modulation (PWM) modules, one or more quadrature encoder inputs (QEI) analogs, one or more 12-bit analog-to-digital converters (ADCs) with 12 analog input channels, details of which are available for the product datasheet.

In an example, the processor may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

It is to be appreciated that the computer system 20063 may include software that acts as an intermediary between users and the basic computer resources described in a suitable operating environment. Such software may include an operating system. The operating system, which can be stored on the disk storage, may act to control and allocate resources of the computer system. System applications may take advantage of the management of resources by the operating system through program modules and program data stored either in the system memory or on the disk storage. It is to be appreciated that various components described herein can be implemented with various operating systems or combinations of operating systems.

A user may enter commands or information into the computer system 20063 through input device(s) coupled to the I/O interface. The input devices may include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processor 20102 through the system bus via interface port(s). The interface port(s) include, for example, a serial port, a parallel port, a game port, and a USB. The output device(s) use some of the same types of ports as input device(s). Thus, for example, a USB port may be used to provide input to the computer system 20063 and to output information from the computer system 20063 to an output device. An output adapter may be provided to illustrate that there can be some output devices like monitors, displays, speakers, and printers, among other output devices that may require special adapters. The output adapters may include, by way of illustration and not limitation, video and sound cards that provide a means of connection between the output device and the system bus. It should be noted that other devices and/or systems of devices, such as remote computer(s), may provide both input and output capabilities.

The computer system 20063 can operate in a networked environment using logical connections to one or more remote computers, such as cloud computer(s), or local computers. The remote cloud computer(s) can be a personal computer, server, router, network PC, workstation, microprocessor-based appliance, peer device, or other common network node, and the like, and typically includes many or all of the elements described relative to the computer system. For purposes of brevity, only a memory storage device is illustrated with the remote computer(s). The remote computer(s) may be logically connected to the computer system through a network interface and then physically connected via a communication connection. The network interface may encompass communication networks such as local area networks (LANs) and wide area networks (WANs). LAN technologies may include Fiber Distributed Data Interface (FDDI), Copper Distributed Data Interface (CDDI), Ethernet/IEEE 802.3, Token Ring/IEEE 802.5, and the like. WAN technologies may include, but are not limited to, point-to-point links, circuit-switching networks like Integrated Services Digital Networks (ISDN) and variations thereon, packet-switching networks, and Digital Subscriber Lines (DSL).

In various examples, the computer system 20063 may comprise an image processor, image-processing engine, media processor, or any specialized digital signal processor (DSP) used for the processing of digital images. The image processor may employ parallel computing with single instruction, multiple data (SIMD) or multiple instruction, multiple data (MIMD) technologies to increase speed and efficiency. The digital image-processing engine can perform a range of tasks. The image processor may be a system on a chip with multicore processor architecture.

The communication connection(s) may refer to the hardware/software employed to connect the network interface to the bus. While the communication connection is shown for illustrative clarity inside the computer system 20063, it can also be external to the computer system 20063. The hardware/software necessary for connection to the network interface may include, for illustrative purposes only, internal and external technologies such as modems, including regular telephone-grade modems, cable modems, optical fiber modems, and DSL modems, ISDN adapters, and Ethernet cards. In some examples, the network interface may also be provided using an RF interface.

Surgical data network associated with the surgical hub system 20060 may be configured as passive, intelligent, or switching. A passive surgical data network serves as a conduit for the data, enabling it to go from one device (or segment) to another and to the cloud computing resources. An intelligent surgical data network includes additional features to enable the traffic passing through the surgical data network to be monitored and to configure each port in the network hub 20061 or network switch 20062. An intelligent surgical data network may be referred to as a manageable hub or switch. A switching hub reads the destination address of each packet and then forwards the packet to the correct port.

Modular devices 1a-1n located in the operating theater may be coupled to the modular communication hub 20065. The network hub 20061 and/or the network switch 20062 may be coupled to a network router 20066 to connect the devices 1a-1n to the cloud computing system 20064 or the local computer system 20063. Data associated with the devices 1a-1n may be transferred to cloud-based computers via the router for remote data processing and manipulation. Data associated with the devices 1a-1n may also be transferred to the local computer system 20063 for local data processing and manipulation. Modular devices 2a-2m located in the same operating theater also may be coupled to a network switch 20062. The network switch 20062 may be coupled to the network hub 20061 and/or the network router 20066 to connect the devices 2a-2m to the cloud 20064. Data associated with the devices 2a-2m may be transferred to the cloud computing system 20064 via the network router 20066 for data processing and manipulation. Data associated with the devices 2a-2m may also be transferred to the local computer system 20063 for local data processing and manipulation.

The wearable sensing system 20011 may include one or more sensing systems 20069. The sensing systems 20069 may include an HCP sensing system and/or a patient sensing system. The one or more sensing systems 20069 may be in communication with the computer system 20063 of a surgical hub system 20060 or the cloud server 20067 directly via one of the network routers 20066 or via a network hub 20061 or network switching 20062 that is in communication with the network routers 20066.

The sensing systems 20069 may be coupled to the network router 20066 to connect to the sensing systems 20069 to the local computer system 20063 and/or the cloud computing system 20064. Data associated with the sensing systems 20069 may be transferred to the cloud computing system 20064 via the network router 20066 for data processing and manipulation. Data associated with the sensing systems 20069 may also be transferred to the local computer system 20063 for local data processing and manipulation.

As illustrated in FIG. 4, the surgical hub system 20060 may be expanded by interconnecting multiple network hubs 20061 and/or multiple network switches 20062 with multiple network routers 20066. The modular communication hub 20065 may be contained in a modular control tower configured to receive multiple devices 1a-1n/2a-2m. The local computer system 20063 also may be contained in a modular control tower. The modular communication hub 20065 may be connected to a display 20068 to display images obtained by some of the devices 1a-1n/2a-2m, for example during surgical procedures. In various aspects, the devices 1a-1n/2a-2m may include, for example, various modules such as an imaging module coupled to an endoscope, a generator module coupled to an energy-based surgical device, a smoke evacuation module, a suction/irrigation module, a communication module, a processor module, a storage array, a surgical device coupled to a display, and/or a non-contact sensor module, among other modular devices that may be connected to the modular communication hub 20065 of the surgical data network.

In one aspect, the surgical hub system 20060 illustrated in FIG. 4 may comprise a combination of network hub(s), network switch(es), and network router(s) connecting the devices 1a-1n/2a-2m or the sensing systems 20069 to the cloud-base system 20064. One or more of the devices 1a-1n/2a-2m or the sensing systems 20069 coupled to the network hub 20061 or network switch 20062 may collect data in real-time and transfer the data to cloud computers for data processing and manipulation. It will be appreciated that cloud computing relies on sharing computing resources rather than having local servers or personal devices to handle software applications. The word “cloud” may be used as a metaphor for “the Internet,” although the term is not limited as such. Accordingly, the term “cloud computing” may be used herein to refer to “a type of Internet-based computing,” where different services—such as servers, storage, and applications—are delivered to the modular communication hub 20065 and/or computer system 20063 located in the surgical theater (e.g., a fixed, mobile, temporary, or field operating room or space) and to devices connected to the modular communication hub 20065 and/or computer system 20063 through the Internet. The cloud infrastructure may be maintained by a cloud service provider. In this context, the cloud service provider may be the entity that coordinates the usage and control of the devices 1a-1n/2a-2m located in one or more operating theaters. The cloud computing services can perform a large number of calculations based on the data gathered by smart surgical instruments, robots, sensing systems, and other computerized devices located in the operating theater. The hub hardware enables multiple devices, sensing systems, and/or connections to be connected to a computer that communicates with the cloud computing resources and storage.

Applying cloud computer data processing techniques on the data collected by the devices 1a-1n/2a-2m, the surgical data network can provide improved surgical outcomes, reduced costs, and improved patient satisfaction. At least some of the devices 1a-1n/2a-2m may be employed to view tissue states to assess leaks or perfusion of sealed tissue after a tissue sealing and cutting procedure. At least some of the devices 1a-1n/2a-2m may be employed to identify pathology, such as the effects of diseases, using the cloud-based computing to examine data including images of samples of body tissue for diagnostic purposes. This may include localization and margin confirmation of tissue and phenotypes. At least some of the devices 1a-1n/2a-2m may be employed to identify anatomical structures of the body using a variety of sensors integrated with imaging devices and techniques such as overlaying images captured by multiple imaging devices. The data gathered by the devices 1a-1n/2a-2m, including image data, may be transferred to the cloud computing system 20064 or the local computer system 20063 or both for data processing and manipulation including image processing and manipulation. The data may be analyzed to improve surgical procedure outcomes by determining if further treatment, such as the application of endoscopic intervention, emerging technologies, a targeted radiation, targeted intervention, and precise robotics to tissue-specific sites and conditions, may be pursued. Such data analysis may further employ outcome analytics processing and using standardized approaches may provide beneficial feedback to either confirm surgical treatments and the behavior of the surgeon or suggest modifications to surgical treatments and the behavior of the surgeon.

Applying cloud computer data processing techniques on the measurement data collected by the sensing systems 20069, the surgical data network can provide improved surgical outcomes, improved recovery outcomes, reduced costs, and improved patient satisfaction. At least some of the sensing systems 20069 may be employed to assess physiological conditions of a surgeon operating on a patient or a patient being prepared for a surgical procedure or a patient recovering after a surgical procedure. The cloud-based computing system 20064 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The operating theater devices 1a-1n may be connected to the modular communication hub 20065 over a wired channel or a wireless channel depending on the configuration of the devices 1a-1n to a network hub 20061. The network hub 20061 may be implemented, in one aspect, as a local network broadcast device that works on the physical layer of the Open System Interconnection (OSI) model. The network hub may provide connectivity to the devices 1a-1n located in the same operating theater network. The network hub 20061 may collect data in the form of packets and sends them to the router in half duplex mode. The network hub 20061 may not store any media access control/Internet Protocol (MAC/IP) to transfer the device data. Only one of the devices 1a-1n can send data at a time through the network hub 20061. The network hub 20061 may not have routing tables or intelligence regarding where to send information and broadcasts all network data across each connection and to a remote server 20067 of the cloud computing system 20064. The network hub 20061 can detect basic network errors such as collisions but having all information broadcast to multiple ports can be a security risk and cause bottlenecks.

The operating theater devices 2a-2m may be connected to a network switch 20062 over a wired channel or a wireless channel. The network switch 20062 works in the data link layer of the OSI model. The network switch 20062 may be a multicast device for connecting the devices 2a-2m located in the same operating theater to the network. The network switch 20062 may send data in the form of frames to the network router 20066 and may work in full duplex mode. Multiple devices 2a-2m can send data at the same time through the network switch 20062. The network switch 20062 stores and uses MAC addresses of the devices 2a-2m to transfer data.

The network hub 20061 and/or the network switch 20062 may be coupled to the network router 20066 for connection to the cloud computing system 20064. The network router 20066 works in the network layer of the OSI model. The network router 20066 creates a route for transmitting data packets received from the network hub 20061 and/or network switch 20062 to cloud-based computer resources for further processing and manipulation of the data collected by any one of or all the devices 1a-1n/2a-2m and wearable sensing system 20011. The network router 20066 may be employed to connect two or more different networks located in different locations, such as, for example, different operating theaters of the same healthcare facility or different networks located in different operating theaters of different healthcare facilities. The network router 20066 may send data in the form of packets to the cloud computing system 20064 and works in full duplex mode. Multiple devices can send data at the same time. The network router 20066 may use IP addresses to transfer data.

In an example, the network hub 20061 may be implemented as a USB hub, which allows multiple USB devices to be connected to a host computer. The USB hub may expand a single USB port into several tiers so that there are more ports available to connect devices to the host system computer. The network hub 20061 may include wired or wireless capabilities to receive information over a wired channel or a wireless channel. In one aspect, a wireless USB short-range, high-bandwidth wireless radio communication protocol may be employed for communication between the devices 1a-1n and devices 2a-2m located in the operating theater.

In examples, the operating theater devices 1a-1n/2a-2m and/or the sensing systems 20069 may communicate to the modular communication hub 20065 via Bluetooth wireless technology standard for exchanging data over short distances (using short-wavelength UHF radio waves in the ISM band from 2.4 to 2.485 GHz) from fixed and mobile devices and building personal area networks (PANs). The operating theater devices 1a-1n/2a-2m and/or the sensing systems 20069 may communicate to the modular communication hub 20065 via a number of wireless or wired communication standards or protocols, including but not limited to Bluetooth, Low-Energy Bluetooth, near-field communication (NFC), Wi-Fi (IEEE 802.11 family), WiMAX (IEEE 802.16 family), IEEE 802.20, new radio (NR), long-term evolution (LTE), and Ev-DO, HSPA+, HSDPA+, HSUPA+, EDGE, GSM, GPRS, CDMA, TDMA, DECT, and Ethernet derivatives thereof, as well as any other wireless and wired protocols that are designated as 3G, 4G, 5G, and beyond. The computing module may include a plurality of communication modules. For instance, a first communication module may be dedicated to shorter-range wireless communications such as Wi-Fi and Bluetooth Low-Energy Bluetooth, Bluetooth Smart, and a second communication module may be dedicated to longer-range wireless communications such as GPS, EDGE, GPRS, CDMA, WiMAX, LTE, Ev-DO, HSPA+, HSDPA+, HSUPA+, EDGE, GSM, GPRS, CDMA, TDMA, and others.

The modular communication hub 20065 may serve as a central connection for one or more of the operating theater devices 1a-1n/2a-2m and/or the sensing systems 20069 and may handle a data type known as frames. Frames may carry the data generated by the devices 1a-1n/2a-2m and/or the sensing systems 20069. When a frame is received by the modular communication hub 20065, it may be amplified and/or sent to the network router 20066, which may transfer the data to the cloud computing system 20064 or the local computer system 20063 by using a number of wireless or wired communication standards or protocols, as described herein.

The modular communication hub 20065 can be used as a standalone device or be connected to compatible network hubs 20061 and network switches 20062 to form a larger network. The modular communication hub 20065 can be generally easy to install, configure, and maintain, making it a good option for networking the operating theater devices 1a-1n/2a-2m.

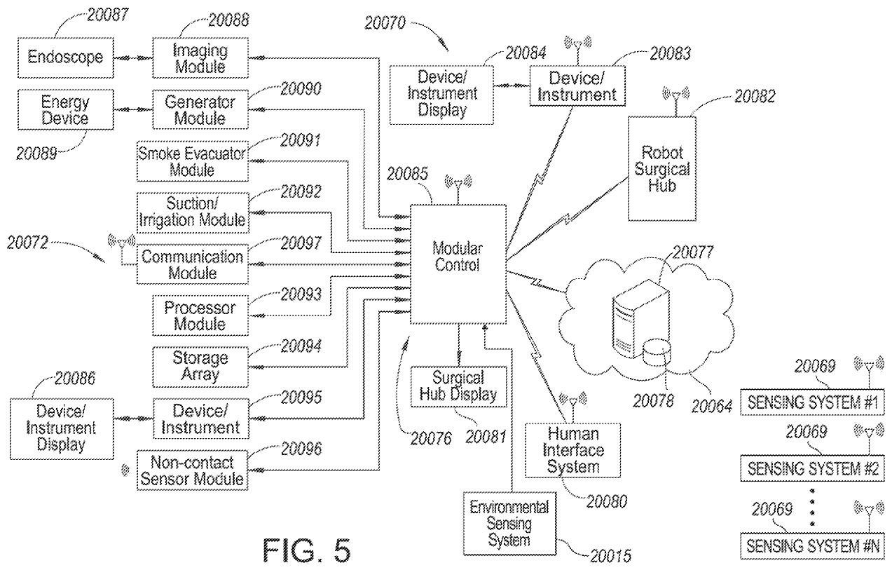

FIG. 5 illustrates a computer-implemented interactive surgical system 20070 that may be a part of the Surgical system 20002. The computer-implemented interactive surgical system 20070 is similar in many respects to the HCP sensing system 20002. For example, the computer-implemented interactive surgical system 20070 may include one or more surgical sub-systems 20072, which are similar in many respects to the Surgical systems 20002. Each sub-surgical system 20072 may include at least one surgical hub 20076 in communication with a cloud computing system 20064 that may include a remote server 20077 and a remote storage 20078. In one aspect, the computer-implemented interactive surgical system 20070 may include a modular control 20085 connected to multiple operating theater devices such as sensing systems 20001, intelligent surgical instruments, robots, and other computerized devices located in the operating theater.

As illustrated in the example of FIG. 5, the modular control 20085 may be coupled to an imaging module 20088 that may be coupled to an endoscope 20087, a generator module 20090 that may be coupled to an energy device 20089, a smoke evacuator module 20091, a suction/irrigation module 20092, a communication module 20097, a processor module 20093, a storage array 20094, a smart device/instrument 20095 optionally coupled to a display 20086 and 20084 respectively, and a non-contact sensor module 20096. The non-contact sensor module 20096 may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

The modular control 20085 may also be in communication with one or more sensing systems 20069 and an environmental sensing system 20015. The sensing systems 20069 may be connected to the modular control 20085 either directly via a router or via the communication module 20097. The operating theater devices may be coupled to cloud computing resources and data storage via the modular control 20085. A robot surgical hub 20082 also may be connected to the modular control 20085 and to the cloud computing resources. The devices/instruments 20095 or 20084, human interface system 20080, among others, may be coupled to the modular control 20085 via wired or wireless communication standards or protocols, as described herein. The human interface system 20080 may include a display sub-system and a notification sub-system. The modular control 20085 may be coupled to a hub display 20081 (e.g., monitor, screen) to display and overlay images received from the imaging module 20088, device/instrument display 20086, and/or other human interface systems 20080. The hub display 20081 also may display data received from devices connected to the modular control 20085 in conjunction with images and overlaid images.

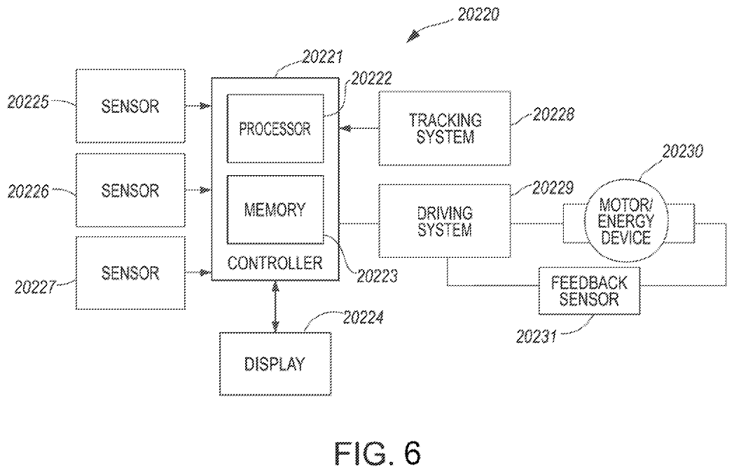

FIG. 6 illustrates a logical diagram of a control system 20220 of a surgical instrument or a surgical tool in accordance with one or more aspects of the present disclosure. The surgical instrument or the surgical tool may be configurable. The surgical instrument may include surgical fixtures specific to the procedure at-hand, such as imaging devices, surgical staplers, energy devices, endocutter devices, or the like. For example, the surgical instrument may include any of a powered stapler, a powered stapler generator, an energy device, an advanced energy device, an advanced energy jaw device, an endocutter clamp, an energy device generator, an in-operating-room imaging system, a smoke evacuator, a suction-irrigation device, an insufflation system, or the like. The system 20220 may comprise a control circuit. The control circuit may include a microcontroller 20221 comprising a processor 20222 and a memory 20223. One or more of sensors 20225, 20226, 20227, for example, provide real-time feedback to the processor 20222. A motor 20230, driven by a motor driver 20229, operably couples a longitudinally movable displacement member to drive the I-beam knife element. A tracking system 20228 may be configured to determine the position of the longitudinally movable displacement member. The position information may be provided to the processor 20222, which can be programmed or configured to determine the position of the longitudinally movable drive member as well as the position of a firing member, firing bar, and I-beam knife element. Additional motors may be provided at the tool driver interface to control I-beam firing, closure tube travel, shaft rotation, and articulation. A display 20224 may display a variety of operating conditions of the instruments and may include touch screen functionality for data input. Information displayed on the display 20224 may be overlaid with images acquired via endoscopic imaging modules.

The microcontroller 20221 may be any single-core or multicore processor such as those known under the trade name ARM Cortex by Texas Instruments. In one aspect, the main microcontroller 20221 may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The microcontroller 20221 may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

The microcontroller 20221 may be programmed to perform various functions such as precise control over the speed and position of the knife and articulation systems. In one aspect, the microcontroller 20221 may include a processor 20222 and a memory 20223. The electric motor 20230 may be a brushed direct current (DC) motor with a gearbox and mechanical links to an articulation or knife system. In one aspect, a motor driver 20229 may be an A3941 available from Allegro Microsystems, Inc. Other motor drivers may be readily substituted for use in the tracking system 20228 comprising an absolute positioning system. A detailed description of an absolute positioning system is described in U.S. Patent Application Publication No. 2017/0296213, titled SYSTEMS AND METHODS FOR CONTROLLING A SURGICAL STAPLING AND CUTTING INSTRUMENT, which published on Oct. 19, 2017, which is herein incorporated by reference in its entirety.

The microcontroller 20221 may be programmed to provide precise control over the speed and position of displacement members and articulation systems. The microcontroller 20221 may be configured to compute a response in the software of the microcontroller 20221. The computed response may be compared to a measured response of the actual system to obtain an “observed” response, which is used for actual feedback decisions. The observed response may be a favorable, tuned value that balances the smooth, continuous nature of the simulated response with the measured response, which can detect outside influences on the system.

The motor 20230 may be controlled by the motor driver 20229 and can be employed by the firing system of the surgical instrument or tool. In various forms, the motor 20230 may be a brushed DC driving motor having a maximum rotational speed of approximately 25,000 RPM. In some examples, the motor 20230 may include a brushless motor, a cordless motor, a synchronous motor, a stepper motor, or any other suitable electric motor. The motor driver 20229 may comprise an H-bridge driver comprising field-effect transistors (FETs), for example. The motor 20230 can be powered by a power assembly releasably mounted to the handle assembly or tool housing for supplying control power to the surgical instrument or tool. The power assembly may comprise a battery which may include a number of battery cells connected in series that can be used as the power source to power the surgical instrument or tool. In certain circumstances, the battery cells of the power assembly may be replaceable and/or rechargeable. In at least one example, the battery cells can be lithium-ion batteries which can be couplable to and separable from the power assembly.

The motor driver 20229 may be an A3941 available from Allegro Microsystems, Inc. A3941 may be a full-bridge controller for use with external N-channel power metal-oxide semiconductor field-effect transistors (MOSFETs) specifically designed for inductive loads, such as brush DC motors. The driver 20229 may comprise a unique charge pump regulator that can provide full (>10 V) gate drive for battery voltages down to 7 V and can allow the A3941 to operate with a reduced gate drive, down to 5.5 V. A bootstrap capacitor may be employed to provide the above battery supply voltage required for N-channel MOSFETs. An internal charge pump for the high-side drive may allow DC (100% duty cycle) operation. The full bridge can be driven in fast or slow decay modes using diode or synchronous rectification. In the slow decay mode, current recirculation can be through the high-side or the low-side FETs. The power FETs may be protected from shoot-through by resistor-adjustable dead time. Integrated diagnostics provide indications of undervoltage, overtemperature, and power bridge faults and can be configured to protect the power MOSFETs under most short circuit conditions. Other motor drivers may be readily substituted for use in the tracking system 20228 comprising an absolute positioning system.

The tracking system 20228 may comprise a controlled motor drive circuit arrangement comprising a position sensor 20225 according to one aspect of this disclosure. The position sensor 20225 for an absolute positioning system may provide a unique position signal corresponding to the location of a displacement member. In some examples, the displacement member may represent a longitudinally movable drive member comprising a rack of drive teeth for meshing engagement with a corresponding drive gear of a gear reducer assembly. In some examples, the displacement member may represent the firing member, which could be adapted and configured to include a rack of drive teeth. In some examples, the displacement member may represent a firing bar or the I-beam, each of which can be adapted and configured to include a rack of drive teeth. Accordingly, as used herein, the term displacement member can be used generically to refer to any movable member of the surgical instrument or tool such as the drive member, the firing member, the firing bar, the I-beam, or any element that can be displaced. In one aspect, the longitudinally movable drive member can be coupled to the firing member, the firing bar, and the I-beam. Accordingly, the absolute positioning system can, in effect, track the linear displacement of the I-beam by tracking the linear displacement of the longitudinally movable drive member. In various aspects, the displacement member may be coupled to any position sensor 20225 suitable for measuring linear displacement. Thus, the longitudinally movable drive member, the firing member, the firing bar, or the I-beam, or combinations thereof, may be coupled to any suitable linear displacement sensor. Linear displacement sensors may include contact or non-contact displacement sensors. Linear displacement sensors may comprise linear variable differential transformers (LVDT), differential variable reluctance transducers (DVRT), a slide potentiometer, a magnetic sensing system comprising a movable magnet and a series of linearly arranged Hall effect sensors, a magnetic sensing system comprising a fixed magnet and a series of movable, linearly arranged Hall effect sensors, an optical sensing system comprising a movable light source and a series of linearly arranged photo diodes or photo detectors, an optical sensing system comprising a fixed light source and a series of movable linearly, arranged photodiodes or photodetectors, or any combination thereof.

The electric motor 20230 can include a rotatable shaft that operably interfaces with a gear assembly that is mounted in meshing engagement with a set, or rack, of drive teeth on the displacement member. A sensor element may be operably coupled to a gear assembly such that a single revolution of the position sensor 20225 element corresponds to some linear longitudinal translation of the displacement member. An arrangement of gearing and sensors can be connected to the linear actuator, via a rack and pinion arrangement, or a rotary actuator, via a spur gear or other connection. A power source may supply power to the absolute positioning system and an output indicator may display the output of the absolute positioning system. The displacement member may represent the longitudinally movable drive member comprising a rack of drive teeth formed thereon for meshing engagement with a corresponding drive gear of the gear reducer assembly. The displacement member may represent the longitudinally movable firing member, firing bar, I-beam, or combinations thereof.

A single revolution of the sensor element associated with the position sensor 20225 may be equivalent to a longitudinal linear displacement d1 of the displacement member, where d1 is the longitudinal linear distance that the displacement member moves from point “a” to point “b” after a single revolution of the sensor element coupled to the displacement member. The sensor arrangement may be connected via a gear reduction that results in the position sensor 20225 completing one or more revolutions for the full stroke of the displacement member. The position sensor 20225 may complete multiple revolutions for the full stroke of the displacement member.

A series of switches, where n is an integer greater than one, may be employed alone or in combination with a gear reduction to provide a unique position signal for more than one revolution of the position sensor 20225. The state of the switches may be fed back to the microcontroller 20221 that applies logic to determine a unique position signal corresponding to the longitudinal linear displacement d1+d2+ . . . dn of the displacement member. The output of the position sensor 20225 is provided to the microcontroller 20221. The position sensor 20225 of the sensor arrangement may comprise a magnetic sensor, an analog rotary sensor like a potentiometer, or an array of analog Hall-effect elements, which output a unique combination of position signals or values.

The position sensor 20225 may comprise any number of magnetic sensing elements, such as, for example, magnetic sensors classified according to whether they measure the total magnetic field or the vector components of the magnetic field. The techniques used to produce both types of magnetic sensors may encompass many aspects of physics and electronics. The technologies used for magnetic field sensing may include search coil, fluxgate, optically pumped, nuclear precession, SQUID, Hall-effect, anisotropic magnetoresistance, giant magnetoresistance, magnetic tunnel junctions, giant magnetoimpedance, magnetostrictive/piezoelectric composites, magnetodiode, magnetotransistor, fiber-optic, magneto-optic, and microelectromechanical systems-based magnetic sensors, among others.

The position sensor 20225 for the tracking system 20228 comprising an absolute positioning system may comprise a magnetic rotary absolute positioning system. The position sensor 20225 may be implemented as an AS5055EQFT single-chip magnetic rotary position sensor available from Austria Microsystems, AG. The position sensor 20225 is interfaced with the microcontroller 20221 to provide an absolute positioning system. The position sensor 20225 may be a low-voltage and low-power component and may include four Hall-effect elements in an area of the position sensor 20225 that may be located above a magnet. A high-resolution ADC and a smart power management controller may also be provided on the chip. A coordinate rotation digital computer (CORDIC) processor, also known as the digit-by-digit method and Volder's algorithm, may be provided to implement a simple and efficient algorithm to calculate hyperbolic and trigonometric functions that require only addition, subtraction, bit-shift, and table lookup operations. The angle position, alarm bits, and magnetic field information may be transmitted over a standard serial communication interface, such as a serial peripheral interface (SPI) interface, to the microcontroller 20221. The position sensor 20225 may provide 12 or 14 bits of resolution. The position sensor 20225 may be an AS5055 chip provided in a small QFN 16-pin 4×4×0.85 mm package.

The tracking system 20228 comprising an absolute positioning system may comprise and/or be programmed to implement a feedback controller, such as a PID, state feedback, and adaptive controller. A power source converts the signal from the feedback controller into a physical input to the system: in this case the voltage. Other examples include a PWIVI of the voltage, current, and force. Other sensor(s) may be provided to measure physical parameters of the physical system in addition to the position measured by the position sensor 20225. In some aspects, the other sensor(s) can include sensor arrangements such as those described in U.S. Pat. No. 9,345,481, titled STAPLE CARTRIDGE TISSUE THICKNESS SENSOR SYSTEM, which issued on May 24, 2016, which is herein incorporated by reference in its entirety; U.S. Patent Application Publication No. 2014/0263552, titled STAPLE CARTRIDGE TISSUE THICKNESS SENSOR SYSTEM, which published on Sep. 18, 2014, which is herein incorporated by reference in its entirety; and U.S. patent application Ser. No. 15/628,175, titled TECHNIQUES FOR ADAPTIVE CONTROL OF MOTOR VELOCITY OF A SURGICAL STAPLING AND CUTTING INSTRUMENT, filed Jun. 20, 2017, which is herein incorporated by reference in its entirety. In a digital signal processing system, an absolute positioning system is coupled to a digital data acquisition system where the output of the absolute positioning system will have a finite resolution and sampling frequency. The absolute positioning system may comprise a compare-and-combine circuit to combine a computed response with a measured response using algorithms, such as a weighted average and a theoretical control loop, that drive the computed response towards the measured response. The computed response of the physical system may take into account properties like mass, inertia, viscous friction, inductance resistance, etc., to predict what the states and outputs of the physical system will be by knowing the input.

The absolute positioning system may provide an absolute position of the displacement member upon power-up of the instrument, without retracting or advancing the displacement member to a reset (zero or home) position as may be required with conventional rotary encoders that merely count the number of steps forwards or backwards that the motor 20230 has taken to infer the position of a device actuator, drive bar, knife, or the like.

A sensor 20226, such as, for example, a strain gauge or a micro-strain gauge, may be configured to measure one or more parameters of the end effector, such as, for example, the amplitude of the strain exerted on the anvil during a clamping operation, which can be indicative of the closure forces applied to the anvil. The measured strain may be converted to a digital signal and provided to the processor 20222. Alternatively, or in addition to the sensor 20226, a sensor 20227, such as, for example, a load sensor, can measure the closure force applied by the closure drive system to the anvil. The sensor 20227, such as, for example, a load sensor, can measure the firing force applied to an I-beam in a firing stroke of the surgical instrument or tool. The I-beam is configured to engage a wedge sled, which is configured to upwardly cam staple drivers to force out staples into deforming contact with an anvil. The I-beam also may include a sharpened cutting edge that can be used to sever tissue as the I-beam is advanced distally by the firing bar. Alternatively, a current sensor 20231 can be employed to measure the current drawn by the motor 20230. The force required to advance the firing member can correspond to the current drawn by the motor 20230, for example. The measured force may be converted to a digital signal and provided to the processor 20222.

For example, the strain gauge sensor 20226 can be used to measure the force applied to the tissue by the end effector. A strain gauge can be coupled to the end effector to measure the force on the tissue being treated by the end effector. A system for measuring forces applied to the tissue grasped by the end effector may comprise a strain gauge sensor 20226, such as, for example, a micro-strain gauge, that can be configured to measure one or more parameters of the end effector, for example. In one aspect, the strain gauge sensor 20226 can measure the amplitude or magnitude of the strain exerted on a jaw member of an end effector during a clamping operation, which can be indicative of the tissue compression. The measured strain can be converted to a digital signal and provided to a processor 20222 of the microcontroller 20221. A load sensor 20227 can measure the force used to operate the knife element, for example, to cut the tissue captured between the anvil and the staple cartridge. A magnetic field sensor can be employed to measure the thickness of the captured tissue. The measurement of the magnetic field sensor also may be converted to a digital signal and provided to the processor 20222.

The measurements of the tissue compression, the tissue thickness, and/or the force required to close the end effector on the tissue, as respectively measured by the sensors 20226, 20227, can be used by the microcontroller 20221 to characterize the selected position of the firing member and/or the corresponding value of the speed of the firing member. In one instance, a memory 20223 may store a technique, an equation, and/or a lookup table which can be employed by the microcontroller 20221 in the assessment.

The control system 20220 of the surgical instrument or tool also may comprise wired or wireless communication circuits to communicate with the modular communication hub 20065 as shown in FIG. 5.

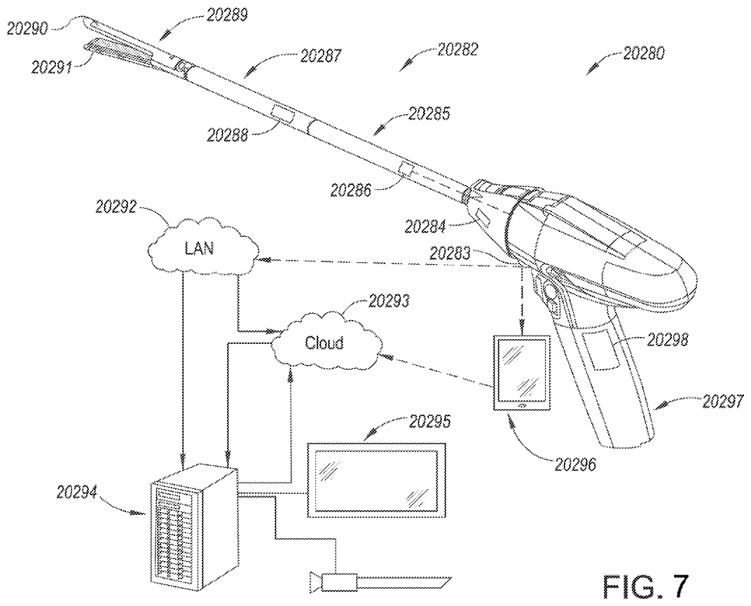

FIG. 7 illustrates an example surgical system 20280 in accordance with the present disclosure and may include a surgical instrument 20282 that can be in communication with a console 20294 or a portable device 20296 through a local area network 20292 and/or a cloud network 20293 via a wired and/or wireless connection. The console 20294 and the portable device 20296 may be any suitable computing device. The surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

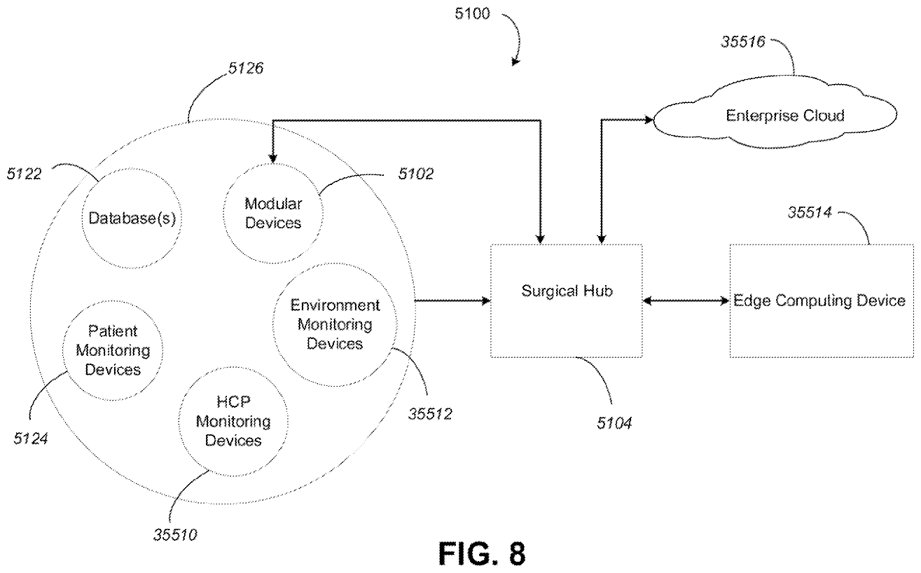

FIG. 8 illustrates a diagram of a situationally aware surgical system 5100, in accordance with at least one aspect of the present disclosure. The data sources 5126 may include, for example, the modular devices 5102 (which can include sensors configured to detect parameters associated with the patient, HCPs and environment and/or the modular device itself), databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The surgical hub 5104 can be configured to derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which is the hardware and/or programming associated with the surgical hub 5104 that derives contextual information pertaining to the surgical procedure from the received data and/or a surgical plan information received from the edge computing system 35514 or an enterprise cloud server 35516.

The situational awareness system of the surgical hub 5104 can be configured to derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. A machine learning system can be trained to accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a further machine learning system, lookup table, or other such system, which generates or retrieves one or more control adjustments for one or more modular devices 5102 when provided the contextual information as input.

A surgical hub 5104 incorporating a situational awareness system can provide a number of benefits for the surgical system 5100. One benefit may include improving the interpretation of sensed and collected data, which would in turn improve the processing accuracy and/or the usage of the data during the course of a surgical procedure. To return to a previous example, a situationally aware surgical hub 5104 could determine what type of tissue was being operated on; therefore, when an unexpectedly high force to close the surgical instrument's end effector is detected, the situationally aware surgical hub 5104 could correctly ramp up or ramp down the motor of the surgical instrument for the type of tissue.

The type of tissue being operated can affect the adjustments that are made to the compression rate and load thresholds of a surgical stapling and cutting instrument for a particular tissue gap measurement. A situationally aware surgical hub 5104 could infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The surgical hub 5104 could then adjust the compression rate and load thresholds of the surgical stapling and cutting instrument appropriately for the type of tissue.

The type of body cavity being operated in during an insufflation procedure can affect the function of a smoke evacuator. A situationally aware surgical hub 5104 could determine whether the surgical site is under pressure (by determining that the surgical procedure is utilizing insufflation) and determine the procedure type. As a procedure type can be generally performed in a specific body cavity, the surgical hub 5104 could then control the motor rate of the smoke evacuator appropriately for the body cavity being operated in. Thus, a situationally aware surgical hub 5104 could provide a consistent amount of smoke evacuation for both thoracic and abdominal procedures.

The type of procedure being performed can affect the optimal energy level for an ultrasonic surgical instrument or radio frequency (RF) electrosurgical instrument to operate at. Arthroscopic procedures, for example, may require higher energy levels because the end effector of the ultrasonic surgical instrument or RF electrosurgical instrument is immersed in fluid. A situationally aware surgical hub 5104 could determine whether the surgical procedure is an arthroscopic procedure. The surgical hub 5104 could then adjust the RF power level or the ultrasonic amplitude of the generator (e.g., “energy level”) to compensate for the fluid filled environment. Relatedly, the type of tissue being operated on can affect the optimal energy level for an ultrasonic surgical instrument or RF electrosurgical instrument to operate at. A situationally aware surgical hub 5104 could determine what type of surgical procedure is being performed and then customize the energy level for the ultrasonic surgical instrument or RF electrosurgical instrument, respectively, according to the expected tissue profile for the surgical procedure. Furthermore, a situationally aware surgical hub 5104 can be configured to adjust the energy level for the ultrasonic surgical instrument or RF electrosurgical instrument throughout the course of a surgical procedure, rather than just on a procedure-by-procedure basis. A situationally aware surgical hub 5104 could determine what step of the surgical procedure is being performed or will subsequently be performed and then update the control algorithms for the generator and/or ultrasonic surgical instrument or RF electrosurgical instrument to set the energy level at a value appropriate for the expected tissue type according to the surgical procedure step.

In examples, data can be drawn from additional data sources 5126 to improve the conclusions that the surgical hub 5104 draws from one data source 5126. A situationally aware surgical hub 5104 could augment data that it receives from the modular devices 5102 with contextual information that it has built up regarding the surgical procedure from other data sources 5126. For example, a situationally aware surgical hub 5104 can be configured to determine whether hemostasis has occurred (e.g., whether bleeding at a surgical site has stopped) according to video or image data received from a medical imaging device. The surgical hub 5104 can be further configured to compare a physiologic measurement (e.g., blood pressure sensed by a BP monitor communicably connected to the surgical hub 5104) with the visual or image data of hemostasis (e.g., from a medical imaging device communicably coupled to the surgical hub 5104) to make a determination on the integrity of the staple line or tissue weld. The situational awareness system of the surgical hub 5104 can consider the physiological measurement data to provide additional context in analyzing the visualization data. The additional context can be useful when the visualization data may be inconclusive or incomplete on its own.

For example, a situationally aware surgical hub 5104 could proactively activate the generator to which an RF electrosurgical instrument is connected if it determines that a subsequent step of the procedure requires the use of the instrument. Proactively activating the energy source can allow the instrument to be ready for use as soon as the preceding step of the procedure is completed.

The situationally aware surgical hub 5104 could determine whether the current or subsequent step of the surgical procedure requires a different view or degree of magnification on the display according to the feature(s) at the surgical site that the surgeon is expected to need to view. The surgical hub 5104 could proactively change the displayed view (supplied by, e.g., a medical imaging device for the visualization system) accordingly so that the display automatically adjusts throughout the surgical procedure.

The situationally aware surgical hub 5104 could determine which step of the surgical procedure is being performed or will subsequently be performed and whether particular data or comparisons between data will be required for that step of the surgical procedure. The surgical hub 5104 can be configured to automatically call up data screens based upon the step of the surgical procedure being performed, without waiting for the surgeon to ask for the particular information.

Errors may be checked during the setup of the surgical procedure or during the course of the surgical procedure. For example, the situationally aware surgical hub 5104 could determine whether the operating theater is setup properly or optimally for the surgical procedure to be performed. The surgical hub 5104 can be configured to determine the type of surgical procedure being performed, retrieve the corresponding checklists, product location, or setup needs (e.g., from a memory), and then compare the current operating theater layout to the standard layout for the type of surgical procedure that the surgical hub 5104 determines is being performed. In some exemplifications, the surgical hub 5104 can compare the list of items for the procedure and/or a list of devices paired with the surgical hub 5104 to a recommended or anticipated manifest of items and/or devices for the given surgical procedure. If there are any discontinuities between the lists, the surgical hub 5104 can provide an alert indicating that a particular modular device 5102, patient monitoring device 5124, HCP monitoring devices 35510, environment monitoring devices 35512, and/or other surgical item is missing. In some examples, the surgical hub 5104 can determine the relative distance or position of the modular devices 5102 and patient monitoring devices 5124 via proximity sensors, for example. The surgical hub 5104 can compare the relative positions of the devices to a recommended or anticipated layout for the particular surgical procedure. If there are any discontinuities between the layouts, the surgical hub 5104 can be configured to provide an alert indicating that the current layout for the surgical procedure deviates from the recommended layout.

The situationally aware surgical hub 5104 could determine whether the surgeon (or other HCP(s)) was making an error or otherwise deviating from the expected course of action during the course of a surgical procedure. For example, the surgical hub 5104 can be configured to determine the type of surgical procedure being performed, retrieve the corresponding list of steps or order of equipment usage (e.g., from a memory), and then compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

Machine learning may be supervised (e.g., supervised learning). A supervised learning algorithm may create a mathematical model from training a dataset (e.g., training data). The training data may consist of a set of training examples. A training example may include one or more inputs and one or more labeled outputs. The labeled output(s) may serve as supervisory feedback. In a mathematical model, a training example may be represented by an array or vector, sometimes called a feature vector. The training data may be represented by row(s) of feature vectors, constituting a matrix. Through iterative optimization of an objective function (e.g., cost function), a supervised learning algorithm may learn a function (e.g., a prediction function) that may be used to predict the output associated with one or more new inputs. A suitably trained prediction function may determine the output for one or more inputs that may not have been a part of the training data. Example algorithms may include linear regression, logistic regression, and neutral network. Example problems solvable by supervised learning algorithms may include classification, regression problems, and the like.

Machine learning may be unsupervised (e.g., unsupervised learning). An unsupervised learning algorithm may train on a dataset that may contain inputs and may find a structure in the data. The structure in the data may be similar to a grouping or clustering of data points. As such, the algorithm may learn from training data that may not have been labeled. Instead of responding to supervisory feedback, an unsupervised learning algorithm may identify commonalities in training data and may react based on the presence or absence of such commonalities in each train example. Example algorithms may include Apriori algorithm, K-Means, K-Nearest Neighbors (KNN), K-Medians, and the like. Example problems solvable by unsupervised learning algorithms may include clustering problems, anomaly/outlier detection problems, and the like

Machine learning may include reinforcement learning, which may be an area of machine learning that may be concerned with how software agents may take actions in an environment to maximize a notion of cumulative reward. Reinforcement learning algorithms may not assume knowledge of an exact mathematical model of the environment (e.g., represented by Markov decision process (MDP)) and may be used when exact models may not be feasible. Reinforcement learning algorithms may be used in autonomous vehicles or in learning to play a game against a human opponent.

Machine learning may be a part of a technology platform called cognitive computing (CC), which may constitute various disciplines such as computer science and cognitive science. CC systems may be capable of learning at scale, reasoning with purpose, and interacting with humans naturally. By means of self-teaching algorithms that may use data mining, visual recognition, and/or natural language processing, a CC system may be capable of solving problems and optimizing human processes.

The output of machine learning's training process may be a model for predicting outcome(s) on a new dataset. For example, a linear regression learning algorithm may be a cost function that may minimize the prediction errors of a linear prediction function during the training process by adjusting the coefficients and constants of the linear prediction function. When a minimal may be reached, the linear prediction function with adjusted coefficients may be deemed trained and constitute the model the training process has produced. For example, a neural network (NN) algorithm (e.g., multilayer perceptrons (MLP)) for classification may include a hypothesis function represented by a network of layers of nodes that are assigned with biases and interconnected with weight connections. The hypothesis function may be a non-linear function (e.g., a highly non-linear function) that may include linear functions and logistic functions nested together with the outermost layer consisting of one or more logistic functions. The NN algorithm may include a cost function to minimize classification errors by adjusting the biases and weights through a process of feedforward propagation and backward propagation. When a global minimum may be reached, the optimized hypothesis function with its layers of adjusted biases and weights may be deemed trained and constitute the model the training process has produced.

Data collection may be performed for machine learning as a first stage of the machine learning lifecycle. Data collection may include steps such as identifying various data sources, collecting data from the data sources, integrating the data, and the like. For example, for training a machine learning model for predicting surgical complications and/or post-surgical recovery rates, data sources containing pre-surgical data, such as a patient's medical conditions and biomarker measurement data, may be identified. Such data sources may be a patient's electronical medical records (EMR), a computing system storing the patient's pre-surgical biomarker measurement data, and/or other like datastores. The data from such data sources may be retrieved and stored in a central location for further processing in the machine learning lifecycle. The data from such data sources may be linked (e.g. logically linked) and may be accessed as if they were centrally stored. Surgical data and/or post-surgical data may be similarly identified, collected. Further, the collected data may be integrated. In examples, a patient's pre-surgical medical record data, pre-surgical biomarker measurement data, pre-surgical data, surgical data, and/or post-surgical may be combined into a record for the patient. The record for the patient may be an EMR.

Data preparation may be performed for machine learning as another stage of the machine learning lifecycle. Data preparation may include data preprocessing steps such as data formatting, data cleaning, and data sampling. For example, the collected data may not be in a data format suitable for training a model. In an example, a patient's integrated data record of pre-surgical EMR record data and biomarker measurement data, surgical data, and post-surgical data may be in a rational database. Such data record may be converted to a flat file format for model training. In an example, the patient's pre-surgical EMR data may include medical data in text format, such as the patient's diagnoses of emphysema, preoperative treatment (e.g., chemotherapy, radiation, blood thinner). Such data may be mapped to numeric values for model training. For example, the patient's integrated data record may include personal identifier information or other information that may identifier a patient such as an age, an employer, a body mass index (BMI), demographic information, and the like. Such identifying data may be removed before model training. For example, identifying data may be removed for privacy reasons. As another example, data may be removed because there may be more data available than may be used for model training. In such case, a subset of the available data may be randomly sampled and selected for model training and the remainder may be discarded.

Data preparation may include data transforming procedures (e.g., after preprocessing), such as scaling and aggregation. For example, the preprocessed data may include data values in a mixture of scales. These values may be scaled up or down, for example, to be between 0 and 1 for model training. For example, the preprocessed data may include data values that carry more meaning when aggregated. In an example, there may be multiple prior colorectal procedures a patient has had. The total count of prior colorectal procedures may be more meaningful for training a model to predict surgical complications due to adhesions. In such case, the records of prior colorectal procedures may be aggregated into a total count for model training purposes.

Model training may be another aspect of the machine learning lifecycle. The model training process as described herein may be dependent on the machine learning algorithm used. A model may be deemed suitably trained after it has been trained, cross validated, and tested. Accordingly, the dataset from the data preparation stage (e.g., an input dataset) may be divided into a training dataset (e.g., 60% of the input dataset), a validation dataset (e.g., 20% of the input dataset), and a test dataset (e.g., 20% of the input dataset). After the model has been trained on the training dataset, the model may be run against the validation dataset to reduce overfitting. If accuracy of the model were to decrease when run against the validation dataset when accuracy of the model has been increasing, this may indicate a problem of overfitting. The test dataset may be used to test the accuracy of the final model to determine whether it is ready for deployment or more training may be required.

Model deployment may be another aspect of the machine learning lifecycle. The model may be deployed as a part of a standalone computer program. The model may be deployed as a part of a larger computing system. A model may be deployed with model performance parameters(s). Such performance parameters may monitor the model accuracy as it is used for predicating on a dataset in production. For example, such parameters may keep track of false positives and false positives for a classification model. Such parameters may further store the false positives and false positives for further processing to improve the model's accuracy.

Post-deployment model updates may be another aspect of the machine learning cycle. For example, a deployed model may be updated as false positives and/or false positives are predicted on production data. In an example, for a deployed MLP model for classification, as false positives occur, the deployed MLP model may be updated to increase the probably cutoff for predicting a positive to reduce false positives. In an example, for a deployed MLP model for classification, as false negatives occur, the deployed MLP model may be updated to decrease the probably cutoff for predicting a positive to reduce false negatives. In an example, for a deployed MLP model for classification of surgical complications, as both false positives and false negatives occur, the deployed MLP model may be updated to decrease the probably cutoff for predicting a positive to reduce false negatives because it may be less critical to predict a false positive than a false negative.

For example, a deployed model may be updated as more live production data become available as training data. In such case, the deployed model may be further trained, validated, and tested with such additional live production data. In an example, the updated biases and weights of a further-trained MLP model may update the deployed MLP model's biases and weights. Those skilled in the art will appreciate that post-deployment model updates may not be a one-time occurrence and may occur as frequently as suitable for improving the deployed model's accuracy.

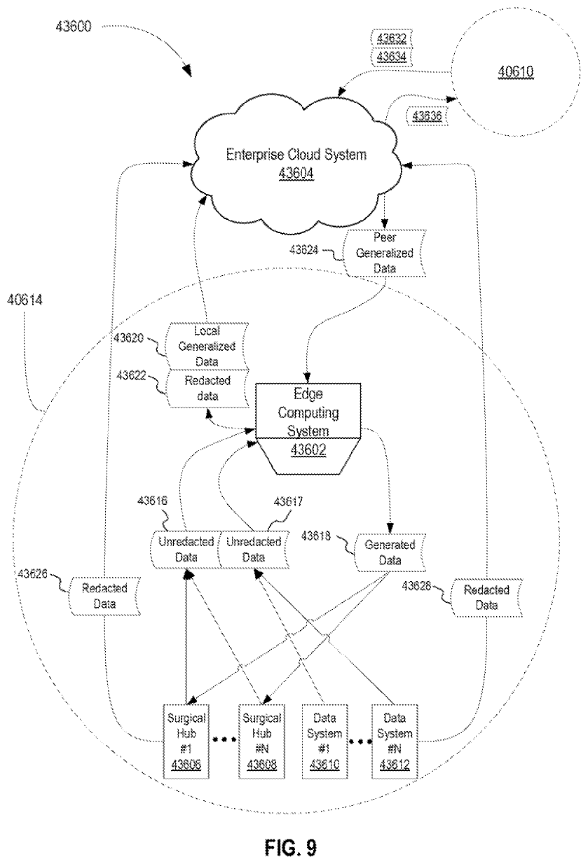

FIG. 9 shows an example multi-level surgical data analysis system 43600. The system 43600 may include multiple levels of systems, such as a lower-level system, a mid-level system, and a higher-level system. A lower-level system may include sub-systems. For example, the sub-systems may include surgical hub #1 (43606) through surgical hub #N (43608), such as surgical hub 2006 as described in FIG. 1A. For example, the sub-systems may include data system #1 (43610) through data system #N (43612). A mid-level system may include an edge computing system 43602. For example, the edge computing system 43602 may be a local cloud computing system that includes a local cloud server and a local cloud storage unit. The higher-level system may include an enterprise cloud system 43604. For example, the enterprise cloud system 43604 may be a cloud computing system 20008 that includes a remote cloud server 20009 and a remote cloud storage unit 20010, as described in FIG. 1A.

The lower-level system and the mid-level system may be co-located on a local data network. For example, surgical hubs 43606 through 43608 and data systems 43610 through 43608 may be co-located with the edge computing system 43602 on a local data network. The local data network may be a local data network of a hospital, such as hospital B (e.g., the medical facility or hospital associated with the edge tier system 40054 in FIG. 1B). The local data network may be within a data boundary. For example, a data boundary 43614 may be defined by rules that patient data privacy are preserved within the boundary. The rules may be health insurance portability and accountability act (HIPAA) related data rules. In an example, a patient's private data may be redacted before being sent outside the boundary. The patient's private data flows without redaction between the edge computing system 43602, and surgical hubs 43606 through 43608 and data systems 43610 through 43608.

The higher-level system may be outside the local data network. For example, the enterprise cloud system 43604 may be outside the data boundary 43614. The enterprise cloud system 43604 may be remote to the edge computing system 43602, and surgical hubs 43606 through 43608 and data systems 43610 through 43608.

The higher-level system may be in communication with more than one local data network. For example, the enterprise cloud system 43604 (e.g., enterprise cloud system 40060 shown in FIG. 1B) may be in communication with the local data network of hospital B that is within the data boundary 43614 (e.g., 40062 shown in FIG. 1B). The enterprise cloud system 43604 may be in communication with the local data network of hospital A (e.g., the medical facility or hospital associated with the edge tier system 40054 in FIG. 1B) that is within a data boundary 40610. For example, the data boundary 40610 may include an edge computing system, surgical hubs, and data systems on the local data network of hospital A.

The lower-level system may provide patient data and clinical data to the mid-level system. For example, surgical hubs 43606 through 43608 in the lower-level system may be in operating room(s) of one or more hospital B's departments, such as the colorectal department, the bariatric department, the thoracic department, or the emergency room (ER) department. One or more of the surgical hubs may provide unredacted data 43616, such as patient personal data and patient clinical data, to the edge computing system 43602.

For example, patient personal data may include a patient's demographics information, such as age, gender, place of residence, occupation, employer, and family status. Patient personal data may include a patient identifier. Patient personal data may be from a patient electronic Medical Record (EMR) database. The interaction between surgical hubs and the EMR database is described in greater detail under the heading of “Data Management and Collection” in U.S. Patent Application Publication No. US 20190206562 A1 (U.S. patent application Ser. No. 16/209,385), titled Method of hub communication, processing, storage and display, filed Dec. 4, 2018, the disclosure of which is herein incorporated by reference in its entirety.

Patient clinical data may include a patient's pre-surgery data (e.g., preoperative data), in-surgery data (e.g., intraoperative data), and post-surgery data (e.g., postoperative data). Preoperative data, intraoperative data, and postoperative data are described in greater detail in FIG. 194's detailed description in U.S. Patent Application Publication No. US 20190206562 A1 (U.S. patent application Ser. No. 16/209,416), titled Method of hub communication, processing, display, and cloud analytics, filed Dec. 4, 2018, the disclosure of which is herein incorporated by reference in its entirety.

Pre-surgery data may include pre-surgery monitoring data. Pre-surgery monitoring data is described in greater detail in U.S. patent application Ser. No. 17/156,318, titled PREDICTION OF ADHESIONS BASED ON BIOMARKER MONITORING, filed Jan. 22, 2021; U.S. patent application Ser. No. 17/156,309, titled PREDICTION OF BLOOD PERFUSION DIFFICULTIES BASED ON BIOMARKER MONITORING, filed Jan. 22, 2021; U.S. patent application Ser. No. 17/156,306, titled PREDICTION OF TISSUE IRREGULARITIES BASED ON BIOMARKER MONITORING, filed Jan. 22, 2021; and U.S. patent application Ser. No. 17/156,321, titled PREDICTION OF HEMOSTASIS ISSUES BASED ON BIOMARKER MONITORING, filed Jan. 22, 2021, the disclosure of which are herein incorporated by reference in its entirety.

In-surgery data may include in-surgery monitoring data. In-surgery monitoring data is described in greater detail in U.S. patent application Ser. No. 17/156,269, titled PRE-SURGICAL AND SURGICAL PROCESSING FOR SURGICAL DATA CONTEXT, filed Jan. 26, 2021, the disclosure of which is herein incorporated by reference in its entirety.

Post-surgery data may include post-surgery monitoring data. Post-surgery monitoring data is described in greater detail in U.S. patent application Ser. No. 17/156,281, titled COLORECTAL SURGERY POST-SURGICAL MONITORING, filed Jan. 22, 2021; U.S. patent application Ser. No. 17/156,272, titled THORACIC POST-SURGICAL MONITORING AND COMPLICATION PREDICTION, filed Jan. 22, 2021; U.S. patent application Ser. No. 17/156,279, titled HYSTERECTOMYSURGERY POST-SURGICAL MONITORING, filed Jan. 22, 2021; U.S. patent application Ser. No. 17/156,284, titled BARIATRIC SURGERY POST-SURGICAL MONITORING, filed Jan. 22, 2021, the disclosure of which are herein incorporated by reference in its entirety.

The lower-level system may provide other patient data to the mid-level system. For example, one of more of the data systems 43610 through 43612 may provide unredacted data 43617 to the edge computing system 43602. One or more data systems may be billing data systems. The unredacted data 43617 may include billing data, payment data, and/or reimbursement data associated with one or more surgical procedures.

For example, the edge computing system 43602 may receive from the lower-level system patient personal data, patient clinical data, and other patient data associated with surgical procedures. Patient clinical data may include pre-surgery data, in-surgery data, and post-surgery data. Other patient data may include billing data, payment data, and reimbursement data. The edge computing system 43602 may perform pre-processing of the received data. For example, the edge computing system 43602 may link patient personal data, patient clinical data, and other patient data using patient identifiers. A data record may be created for a surgical procedure associated with a patient. For example, a data record may include the patient's personal data. For example, a data record may include the surgical procedure's pre-surgery data, in-surgery data, and/or post-surgery data. For example, a data record may include the surgical procedure's billing data, payment data, and/or reimbursement data.

A data record (e.g., a linked data record) may be associated with a surgical procedure type. For example, a linked data record may be created for a colorectal surgical procedure (e.g., a laparoscopic sigmoid colectomy procedure). For example, a linked data records may be created for a bariatric surgical procedure (e.g., a laparoscopic sleeve gastrectomy). For example, a linked data record may be created for a thoracic surgical procedure (e.g., a lung segmentectomy procedure).

A surgical procedure may include surgical steps. For example, a laparoscopic sigmoid colectomy procedure may include the following surgical steps: initiate, access, mobilize colon, resect sigmoid, perform anastomosis, and conclude.

A surgical step may include surgical tasks. For example, the surgical step “initiate” for a laparoscopic sigmoid colectomy procedure may include the following surgical tasks: make incisions, place trocars, and assess adhesions. For example, the surgical step “access” may include the following surgical tasks: dissect adhesions, dissect mesentery, and identify ureter.

A surgical task may include a surgical instrument selection and surgical choices. For example, in surgical step “initiate” of the laparoscopic sigmoid colectomy procedure, the surgical task “make incisions” (e.g., for trocar placement) may include a surgical instrument selection 33016 of scalpel. The surgical task may include a surgical choice of incision length of 10 mm for a laparoscope port. The surgical task may include a surgical choice of incision location of umbilicus for a laparoscope port. The surgical task may include a surgical choice of incision length of 5 mm for a grasper port. The surgical task may include a surgical choice of incision location of upper right quadrant of abdomen for a grasper port. The surgical task may include a surgical choice of incision length of 5 mm for a harmonic energy device port. The surgical task may include a surgical choice of incision location of lower right quadrant of abdomen for a harmonic energy device port.

For example, the surgical task “dissect mesentery” in the surgical step “access” of the laparoscopic sigmoid colectomy procedure may include a surgical instrument selection of grasper. The surgical task may include a surgical instrument selection of a harmonic energy device. The surgical task may include a surgical choice of performing dissection in the direction of medial-to-lateral. The surgical task may include a surgical choice of performing dissection in the direction of lateral-to-medial.

The surgical steps, surgical tasks, surgical choices, surgical instrument selection, and post-surgery care choices described herein may be a part of a surgical procedure plan.

A surgical procedure plan may include post-surgery care choices. In examples, a post-surgery care choice may be length of stay before discharge, duration of ventilator use, intensive care unit (ICU) monitoring, or performing a spirometry test.

The edge computing system 43602 may create the linked data records in its memory for further processing. The edge computing system 43602 may store the linked data records in a datastore for further processing.

The linked data records may be further processed. For example, the linked data records may be split into subsets and one subset of the linked data records may be further processed. In an example, a subset A of the linked data records may be the records associated with a laparoscopic sigmoid colectomy procedure and where the respective post-surgery data portion of each of the records indicates there were no post-surgery complication(s) or readmission(s).

Subset A of the linked data records may be further processed in preparation for training a machine learning model A. For example, a new data field may be created and appended to each data record of subset A. The new data field may be derived from billing data and reimbursement data of each data record. For example, a new data field may indicate whether a surgical procedure has a reimbursement rate of at least 80% or not. A reimbursement rate of at least 80% of billed amount for medical services provided may be a typical reimbursement rate in the health care industry.

The new data field may serve as each subset A data record's label for training model A using a supervised machine learning algorithm (e.g., a neutral network or a decision tree algorithm). Those of skill in the art will appreciate any suitable machine learning algorithm may be used for training model A. Machine learning algorithms are described in greater detail in U.S. patent application Ser. No. 17/156,293, titled MACHINE LEARNING TO IMPROVE ARTIFICIAL INTELLIGENCE ALGORITHM ITERATIONS, filed Jan. 22, 2021.

In an example, when model A is deemed suitably trained, one or more patterns may be detected in the model (e.g., decision points detected using a decision tree algorithm). An example pattern (“pattern #1”) may be that the laparoscopic sigmoid colectomy procedures with no post-surgery complication(s) or readmission(s) and with the following additional characteristics have a reimbursement rate of at least 80%: (1) they were performed on patients that are 20-45 in age, male, with no pre-conditions, and no prior surgeries in the past; (2) there were no in-surgery complication(s); and (3) the post-surgery length of stay before discharge is two days or more. The characteristics may be decision points in the decision tree from model A. An implication of pattern #1 is that for a laparoscopic sigmoid colectomy procedure with no post-surgery complication(s) or readmission(s) and with characteristics (1) and (2), reducing a post-surgery length of stay from more than two days to two days, there may be no decline in quality of clinical outcome or in reimbursement rate for the medical facility in question (e.g., hospital B).

An example pattern (“pattern #2”) may be that the laparoscopic sigmoid colectomy procedures with no post-surgery complication(s) or readmission(s) and with the following additional characteristics have a reimbursement rate of at least 80%: (1) they were performed on patients that are 20-45 in age, male, with at least one pre-condition, and with at least one prior colorectal surgery in the past; (2) there were no in-surgery complication(s); and (3) the post-surgery length of stay before discharge is four days or more. An implication of pattern #2 is that for a laparoscopic sigmoid colectomy procedure with no post-surgery complication(s) or readmission(s) and with characteristics (1) and (2), reducing a post-surgery length of stay from more than four days to four days, there may be no decline in quality of clinical outcome or in reimbursement rate for the medical facility in question (e.g., hospital B).

For example, a subset B of the linked data records may be split from the linked data records and further processed. Subset B of the linked data records may be the records associated with a laparoscopic sigmoid colectomy procedure and where either the respective in-surgery data portion or the respective post-surgery data portion of the each of the records indicates at least one in-surgery complication or at least one post-surgery complication, respectively.

Subset B of the linked data records may be further processed in preparation for training a machine learning model B. For example, a new data field may be created and appended to each data record of subset B. The new data field may be derived from billing data and reimbursement data of each data record. For example, a new data field may indicate whether a surgical procedure has a denied claim or not.

The new data field may serve as each subset B data record's label for training model B using a supervised machine learning algorithm (e.g., a neutral network or a decision tree algorithm). Those of skill in the art will appreciate any suitable machine learning algorithm may be used for training model B.

In an example, when model B is deemed suitably trained, one or more patterns may be detected in the model (e.g., decision points detected using a decision tree algorithm). An example pattern (“pattern #3”) may be that the laparoscopic sigmoid colectomy procedures with at least one in-surgery complication or at least one post-surgery complication and with the following additional characteristics have a denied claim for a medical procedure performed: (1) they were performed on patients that are 20-45 in age, male, with no pre-conditions, and no prior surgeries in the past; (2) an in-surgery complication of at least one damaged ureter; and (3) a sharp dissection tool is used in the access step's dissect mesentery surgical task. The characteristics may be decision points in the decision tree from model B. An implication of pattern #3 is that for a laparoscopic sigmoid colectomy procedure with characteristics (1), if a dull dissection tool is used instead of the sharp dissection tool, the in-surgery complication of damaged ureter(s) may be prevented, and the denied claim may be prevented. Accordingly, there may be both an improvement in the quality of clinical outcome and an improvement in reimbursement amount (e.g., for the medical facility in question (e.g., hospital B)).

An example pattern (“pattern #4”) may be that the laparoscopic sigmoid colectomy procedures with at least one in-surgery complication or at least one post-surgery complication and with the following additional characteristics have a denied claim for a medical procedure performed: (1) they were performed on patients that are 20-45 in age, male, with no pre-conditions, and no prior surgeries in the past; (2) an in-surgery complication of at least one damaged ureter; and (3) a surgical choice made to not identify ureter before dissecting mesentery in the access step's dissect mesentery surgical task. The characteristics may be decision points in the decision tree from model B. An implication of pattern #4 is that for a laparoscopic sigmoid colectomy procedure with characteristics (1), if a surgical choice is made to identify ureter before dissecting mesentery, the in-surgery complication of damaged ureter(s) may be prevented, and the denied claim may be prevented. Accordingly, there may be both an improvement in the quality of clinical outcome and an improvement in reimbursement amount (e.g., for the medical facility in question (e.g., hospital B)).

An example pattern (“pattern #5”) may be that the laparoscopic sigmoid colectomy procedures with at least one in-surgery complication or at least one post-surgery complication and with the following additional characteristics have a denied claim for a medical procedure performed: (1) they were performed on patients that are 20-45 in age, male, with no pre-conditions, and no prior surgeries in the past; (2) an in-surgery complication of at least one damaged ureter; and (3) the ultrasonic device's (dissection tool) maximum period for energy application is above a threshold T. The characteristics may be decision points in the decision tree from model B. An implication of pattern #5 is that for a laparoscopic sigmoid colectomy procedure with characteristics (1), if the ultrasonic device's maximum period for energy application is below threshold T, potential lateral thermo damage may be reduced. Accordingly, the in-surgery complication of damaged ureter(s) may be prevented and the denied claim may be prevented. Accordingly, there may be both an improvement in the quality of clinical outcome and an improvement in reimbursement amount (e.g., for the medical facility in question (e.g., hospital B)).

The edge computing system 43602 may create generated data 43618. The generated data 43618 may include a suggestion for surgical choice in a surgical procedure plan, a suggestion for post-surgery care choice in a surgical procedure plan, a suggestion for a surgical instrument selection, or an operating parameter adjustment for a selected surgical instrument.

For example, the edge computing system 43602 may create generated data 43618 based on the patterns detected in machine learning models as described. In an example, generated data 43618 may be a suggestion for a post-surgery care choice in a surgical procedure plan based on pattern #1. The suggestion may be that for any future laparoscopic sigmoid colectomy procedure if the procedure's patient personal data, patient clinical data, and other patient data match pattern #1, a post-surgery care choice may include two days (and not more than two days) of post-surgery stay before discharge.

In an example, generated data 43618 may be a suggestion for a post-surgery care choice in a surgical procedure plan based on pattern #2. The suggestion may be that for any future laparoscopic sigmoid colectomy procedure if the procedure's patient personal data, patient clinical data, and other patient data match pattern #2, the associated surgical procedure plan includes a post-surgery care choice of four days (and not more than four days) of post-surgery stay before discharge.

In an example, generated data 43618 may be a suggestion for a surgical instrument selection in a surgical procedure plan based on pattern #3. The suggestion may be that for any future laparoscopic sigmoid colectomy procedure if the procedure's patient personal data, patient clinical data, and other patient data match pattern #3, a dull dissection tool may be a selected surgical instrument for the access step's dissect mesentery surgical task in the associated surgical procedure plan.

In an example, generated data 43618 may be a suggestion for a surgical choice in a surgical procedure plan based on pattern #4. The suggestion may be that for any future laparoscopic sigmoid colectomy procedure if the procedure's patient personal data, patient clinical data, and other patient data match pattern #4, a surgical choice of identifying ureter before dissecting mesentery may be included as part of the access step's dissect mesentery surgical task in the associated surgical procedure plan.

In an example, generated data 43618 may be an operational parameter adjustment of a surgical instrument selected in a surgical instrument plan based on pattern #5. The adjustment may be that for any future laparoscopic sigmoid colectomy procedure if the procedure's patient personal data, patient clinical data, and other patient data match pattern #5, a control program update is generated to reduce a selected ultrasonic device's maximum period for energy application to below threshold T.

The edge computing system 43602 may create generated data 43618 automatically. For example, after machine learning models (e.g., models A and B) are trained, the edge computing system 43602 may create generated data 43618 without a request for it. The automatically created generated data 43618 and associated trained model may be sent to one or more of surgical hubs #1 (43606) through surgical hub #N (43608) to optimize a surgical procedure's clinical outcome and/or cost effectiveness.

For example, the suggestions based on pattern #1 through pattern #4 may be implemented as computer-executable instructions (e.g., scripts, executables, and the like) and sent with the respective trained model to one or more of surgical hubs #1 through #N. The suggestions and the respective models may be stored on the surgical hubs. In an example, when a surgeon is planning a laparoscopic sigmoid colectomy procedure (e.g., on a surgical procedure planning interface coupled with a surgical hub), the trained models may be executed using a laparoscopic sigmoid colectomy procedure's associated data, including patient personal data, patient clinical data, and other patient data as input. If the input data matches the detected pattern associated with a stored suggestion and the trained model predicts an output that corresponds with the pattern, the stored suggestion may be retrieved and presented. For example, if the input data matches pattern #1 and model A predicts a reimbursement rate of at least 80% using data associated with the laparoscopic sigmoid colectomy procedure under planning, the suggestion for a post-surgery care choice of two days may be presented.

For example, the operational parameter adjustment based on pattern #5 may be implemented as computer-executable instructions (e.g., script(s), executable(s), and the like) and sent with the respective trained model to one or more of surgical hubs #1 through #N. The adjustment and the respective model may be stored on the surgical hubs. In an example, when a surgeon is planning a laparoscopic sigmoid colectomy procedure (e.g., on a surgical procedure planning interface coupled with a surgical hub), the trained models may be executed using a laparoscopic sigmoid colectomy procedure's associated data, including patient personal data, patient clinical data, and other patient data as input. If the input data matches the detected pattern associated with a stored adjustment and the trained model predicts the output that corresponds with the pattern, the adjustment is retrieved and sent to the target surgical instrument when the instrument becomes linked to the surgical hub. For example, if the input data matches pattern #5 and model B predicts a denied claim using data associated with the laparoscopic sigmoid colectomy procedure under planning, a control program update associated with reducing a selected ultrasonic device's maximum period for energy application to below threshold T may be retrieved and sent to the target surgical instrument linked to the surgical hub.

The edge computing system 43602 may create generated data 43618 upon request. For example, after machine learning models (e.g., models A and B) are trained, the edge computing system 43602 may create generated data 43618 upon a request. In an example, the suggestions based on pattern #1 through pattern #4 may be implemented as application programming interface (APIs) on the edge computing system 43602. When a surgeon is planning a laparoscopic sigmoid colectomy procedure (e.g., on a surgical procedure planning interface coupled with a surgical hub), the surgical hub may invoke an API on the edge computing system 43602 with a data record that includes the laparoscopic sigmoid colectomy procedure's associated data as input, including patient personal data, patient clinical data, and other patient data. When the API is invoked, the trained models may be executed using the input. If the input data matches the detected pattern associated with a stored suggestion and the associated trained model predicts the output that corresponds with the pattern, the stored suggestion is sent back as an API response to the surgical hub. For example, if the input data matches pattern #1 and model A predicts a reimbursement rate of at least 80% using the input data, the suggestion for a post-surgery care choice of two days may be send back as an API response to the surgical hub.

The edge computing system 43602 may send local generalized data 43620 to enterprise cloud system 43604. For example, the local generalized data 43620 may include trained machine learning model(s), such as model A or model B described herein. For example, the local generalized data 43620 may include generated data 43618 described herein that are associated with the trained machine learning models.

The edge computing system 43602 may receive peer generalized data 43624 from the enterprise cloud system 43604. For example, peer generalized data may be the local generalized data 43620 as described herein that have been further processed. For example, models trained by the edge computing system 43602 (e.g., model A and/or model B) may be sent as generalized data 43636 to a second edge computing system at a second medical facility or hospital (e.g., hospital A in data boundary 40610). The models may be further trained by the second edge computing system using data associated with laparoscopic sigmoid colectomy procedures in data boundary 40610. The further trained models may be used at the second medical facility or hospital. The further trained models may be sent as generalized data 43632 back to enterprise cloud system 43604. The further trained models may be sent as a part of the peer generalized data 43624 to other medical facilities or hospitals, such as the edge computing system 43602 in data boundary 43614.

In response to receiving the peer generalized data 43624, the edge computing system 43602 may further process the peer generalized data 43624. For example, the further trained models included in the peer generalized data 43624 may be further trained using data associated with laparoscopic sigmoid colectomy procedures in data boundary 40614, such as the training of model A or model B by the edge computing system 43602 described herein. Accordingly, the generated data 43618 may be recreated based on the further trained models and sent to one or more of surgical hubs 43606 through 43608. The local generalized data 43620 may be recreated based on the further trained models and the updated generated data 43618, and sent to the enterprise cloud system 43604.

Systems at a medical facility or hospital may send redacted data to enterprise cloud system 43604. For example, the edge computing system 43602 may send redacted data 43622 to enterprise cloud system 43604. In an example, the redacted data 43622 may be output data from further processing the unredacted 43616 (e.g., patient personal data and patient clinical data). The further processing may be to strip patient private information from the unredacted 43616. The patient private information may be age, employer, body mass index (BMI), or any data that can be used to ascertain the identity of a patient. The redaction process is described in greater detail under the heading of “Data Management and Collection” in U.S. Patent Application Publication No. US 20190206562 A1 (U.S. patent application Ser. No. 16/209,385), titled Method of hub communication, processing, storage and display, filed Dec. 4, 2018, the disclosure of which is herein incorporated by reference in its entirety.

For example, surgical hubs #1 through N may send redacted data 43626 to the enterprise cloud system 43604. Data systems #1 through N may send redacted data 43626 to the enterprise cloud system 43604.

For example, the edge computing system in data boundary 40610 may send redacted data 43634 (e.g., similar to redacted data 43622) to the enterprise cloud system 43604.

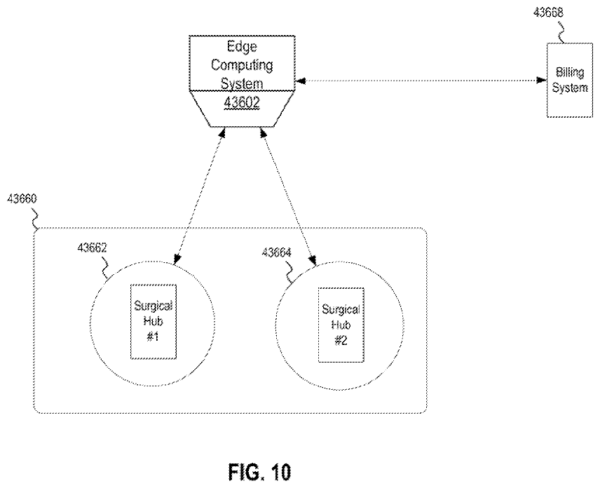

An edge computing network may be an edge cloud system (e.g., the edge computing system 43602 in FIG. 9). The edge cloud system may communicate with a cloud system (e.g., remote server(s), such as the enterprise cloud system 43604 in FIG. 9) and the intra-OR hub network (e.g., the interactive hub-to-hub intra-network 43660 in FIG. 10.

Local facility and/or network aggregation of in-network full patient data records (e.g., unredacted data 43616 and unredacted 43617 in FIG. 9) may be performed. Local facility and/or network outcome aggregation may be performed. The edge computing system's processing and data storage may be within a data network of a treatment facility (e.g., hospital B described in FIG. 9). The edge computing system may use expanded patient data. The expanded patient data may be linked to other intra-network systems (e.g., data systems 43610 through 43612 in FIG. 9), such as a billing system, an ordering and supply system, a clinical system, and/or a laboratory system. A billing system may include reimbursement data, patient payment data, and other data. An order and supply system may include cost of product data, utilization rate of product vs. waste data, on-hand stack and delivery frequency data. A clinical system may include outcomes data, readmittance rate data, time in the hospital data, infection rate data, and ER room visits data. A laboratory system may include frequency of testing data, efficacy of the test on outcomes of the patient data, speed or processing and lab backlog data, and/or lab costs data.

Balancing treatments and costs for a specific facility may be performed. Treatment improvements may be determined. For example, improved treatments may be used as a default procedure(s) and treatment regime(s) for a surgeon and/or doctor to start a surgical procedure plan from (e.g., generated data 43618 in FIG. 9). Value analysis of outcomes may be performed (e.g., for surgical procedure planning). For example, machine learning may be used to determine optimized combinations of treatments and procedure for value-based outcomes of the patient (e.g., as described in FIG. 9). For example, cost and/or outcome changes may be highlighted based on the proposed deviations from the machine learning and/or artificial intelligence (AI)-derived optimized combinations of treatments and procedure for value-based outcomes of the patient.

Staff and OR utilizations may be optimized. For example, a mix of surgeons may be determined to drive the profit of departments to hospital usage. For example, scheduling and staffing surge may be tracked to determine optimized staff utilizations.

Advance imaging or other supplementation of surgical procedure may be determined for value and/or outcome improvements. For example, the number of robots to purchase may be determined to balance OR usage to patient throughput.

The local small cloud system (e.g., the edge cloud system/edge computing system) may enable the facility limited machine learning using full patient records (e.g., unredacted data 43616 and unredacted 43617 in FIG. 9).

Some cloud system (e.g., the enterprise cloud system 43604 in FIG. 9) may perform analysis using redacted and anonymized systems, such as longitudinal databases. The cloud system may perform global analyses. The global analyses may use larger subject populations (e.g., millions) and may lack the resolution of specific costs and/or specific treatments. The global analyses be useful for changes in best-practices, new reimbursement codes, etc. Longitudinal databases used in the global analyses may track specific patients through time to analyze complications, medication, and/or patient response to treatments. The data in the longitudinal databases may be anonymized (e.g., redacted). The anonymized data may not provide needed information to provide a hospital or facility specific recommendations for balancing their facility aspects and costs. The longitudinal databases may be a bariatric outcomes longitudinal database (BOLD). The longitudinal databases may be MarketScan. The types of data that may be analyzed using the longitudinal databases are recoveries and complications, biometrics before and after, and/or medication usage.

Analysis by the edge cloud system may compare specific billing to one of more of patient outcomes, most successful reimbursement and reimbursement code usage, and/or best starting point procedures to be most successful for reimbursement with lowest cost, staffing skill needs, staffing utilization and OR usage, etc.

The edge cloud may supply anonymized datasets (e.g., the redacted data 43622 in FIG. 9), generalized conclusions (e.g., local generalized data 43620 in FIG. 9), and product specific data to systems outside of the HIPAA protected networks (e.g., data boundary 40614 in FIG. 9). The data-sharing may allow the facility to use machine learning to improve their operational parameters and outcomes (e.g., as generated data 43618 is generated using machine learning, as described in FIG. 9). The edge cloud may share the anonymized data to a remote cloud system (e.g., a remote cloud server, such as the enterprise cloud system 43604 in FIG. 9). The remote cloud system may build more global, instrumentation, and/or treatment conclusions.

FIG. 10 is a block diagram of an example edge computing system operating with an intra-surgical hub network. Surgical hub(s) may be in different ORs. The OR surgical hub(s) may form an intra-network. The OR surgical hub(s) may be coupled to an edge computing network and/or system (e.g., edge computing system 43602 shown in FIG. 9) to create an interactive hub-to-hub intra-network. In examples, surgical hub #1 and surgical hub #2 (e.g., surgical hubs 43606 through 43608 in FIG. 9) may be in OR 43662 and OR 43664, respectively. Surgical hub #1 and surgical hub #2 may be coupled with the edge computing system 43602 to create an interactive hub-to-hub intra-network 43660.

The edge computing system 43602 may create leverageable competitive data systems over their competitor networks (e.g., competitor treatment networks). The edge computing system may be within a medical facility's (e.g., hospital B described in FIG. 9) confidential network (e.g., the data boundary 40614 in FIG. 9). The HIPAA data from patient(s) may be used in combination with clinical outcomes data to determine treatments (e.g., new or value-added treatments).

For example, a procedure's reimbursement rate may be used with the outcomes data to instruct surgical procedure plan(s) and/or recovery plan(s). In an example, reimbursement data from billing system 43668 (e.g., a system from data systems 43610 through 43612 in FIG. 9) and outcomes data from surgical hub #1 and surgical hub #2 in OR 43662 and OR 43664, respectively, may be balanced to identify value-added treatments as a starting point for surgical procedure plan(s) and/or recovery plan(s). In an example, a treatment or a recovery plan may deviate from the starting point (e.g., a value-added plan or treatment), the impacts on outcomes, probabilities of complication, and/or cost may be determined for the deviation. In an example, when value-added treatments result from a combination of secondary costs (e.g., duration of hospital stay, hospital acquired infection treatment, readmission rates, and/or emergency services secondary rates), the data may be used to support changes in the value-added treatment reimbursement categorization, or indications for use expansion.

For example, when a surgeon is within the intra-network 43660 (e.g., when operating on surgical hub #1), the surgeon may build or rely on the value-added treatment(s) of another surgeon (e.g., from surgical hub #2) within the intra-network 43660 to increase efficiency, outcomes or low complication rates.

The edge computing system 43602 may suggest changes to other systems (e.g., (e.g., a system from data systems 43610 through 43612 or surgical hubs 43606 through 43608 in FIG. 9) such as, a billing system, an ordering system, a sterilization system, etc. The change(s) may increase the value for the facility for good outcomes. For example, the change(s) to the payment system may be leveraged and may allow patients to pay for outcomes and share in the cost and savings of treatment adjustments. The treatment adjustments may help a facility (e.g., a medical facility) adjust staffing, utilization rate, order timing, etc. For example, surgical procedures may be scheduled with patient treatment outcomes and low burden on the health care facility to balance surge capacity needs.

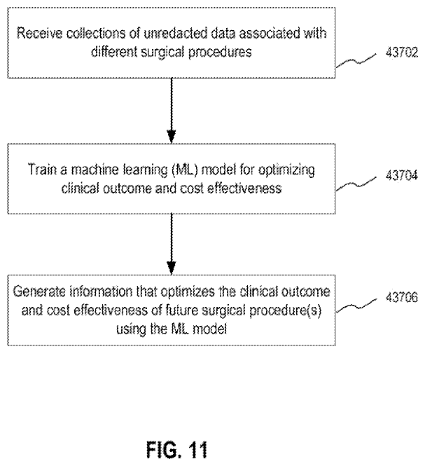

FIG. 11 is the flow chart of an example operation of an edge computing system. The edge computing system may be the edge computing system 43604 described in FIG. 9.

At 43702, collections of unredacted data associated with different surgical procedures may be received. For example, a first collection of unredacted data associated with a first surgical procedure may be received. For example, a second collection of respective unredacted data associated with a second surgical procedure may be received. The first surgical procedure and the second surgical procedure may be past surgical procedures. The first collection of unredacted data and the second collection of unredacted data may be received from at least one of a surgical hub or a data system on a local data network. The first collection of unredacted data or the second collection of unredacted data may include patient personal data, patient clinical data, and other patient data. The local data network may be within a boundary protected by health insurance portability and accountability act (HIPAA) data rules.

For example, the patient personal data may include a patient identifier. The patient clinical data may include a patient identifier. The other patient data may include a patient identifier.

For example, the patient personal data may include one or more of demographics information, such as age, gender, place of residence, occupation, or family status. For example, the patient clinical data includes one or more of pre-surgery data, in-surgery data, or post-surgery data. For example, the other data may include one or more of billing data, payment data, or reimbursement data.

At 43704, a machine learning (ML) model may be trained for optimizing clinical outcome and cost effectiveness of future surgical procedure(s) using the collections of unredacted data associated with different surgical procedures For example, the future surgical procedure(s) may include a third surgical procedure. The third surgical procedure may be a same type of surgical procedure as the first surgical procedure and the second surgical procedure.

At 43706, information that optimizes the clinical outcome and cost effectiveness of future surgical procedure(s) using the ML model may be generated. For example, the information that optimizes the clinical outcome and cost effectiveness of the third surgical procedure may include one or more of aspects of a surgical procedure plan associated with the third surgical procedure, such as a surgical choice, a surgical instrument selection, or a post-surgery care choice. For example, the information that optimizes the clinical outcome and cost effectiveness of the third surgical procedure may include an operational parameter for a surgical instrument associated with the surgical instrument selection.

The information that optimizes the clinical outcome and cost effectiveness of future surgical procedure(s) may be sent to a surgical hub from the at least one of a surgical hub or a data system. The information that optimizes the clinical outcome and cost effectiveness of future surgical procedure(s) may be sent to a surgical procedure planning user interface.

For example, a request for the information that optimizes the clinical outcome and cost effectiveness of future surgical procedure(s) may be received. For example, the request may be from the surgical hub from the at least one of a surgical hub or a data system. For example, in response, the information may be sent to the surgical hub.

For example, the information that optimizes the clinical outcome and cost effectiveness of future surgical procedure(s) may be sent to a cloud computing system.

For example, the first collection of unredacted data may be redacted. The second collection of unredacted data may be redacted. The redacted first collection of unredacted data and the redacted second collection of unredacted data may be sent to the cloud computing system.

Predictive maintenance of an individual hub system or node may be directed by an edge cloud system. An edge cloud system may be defined as an edge computing system concentric to a facility's gateway to a cloud system and acts as a sub-cloud system, e.g., with only the in-network interactions to react to and/or draw from. The edge cloud is within the HIPAA controlled private data network. The edge cloud may act on data and interactions of hubs that for privacy reasons. The data and interactions may not be shared with systems outside of their network.

The edge cloud system may monitor each of the hub systems for security, data storage capacity, and errors. As a hub system reaches a predefined timing, a predefined utilization of resources, or a number of detected errors (e.g., numbers of reboots, communication errors, out of date software, etc.), it may schedule and may initiate a maintenance activity. If the maintenance is beyond an automated check, a notification to service personal and administration may be flagged. If the hub system is flagged for manual maintenance, the hub system may be automatically swapped with another hub system that may automatically be configured and downloaded with all the information from the out-for-service hub system, e.g., to make interaction in the operating room (OR). If the errors detected are during a procedure the hub system may notify users in the OR of the issue and go into a limp mode. The limp mode may be where the system shuts off all non-room critical functions to avoid error propagation and allows for the completion of the procedure before being backed up and taken out of service.

Each hub system may have a standard interaction cadence of reporting function, local analysis of the attached systems, usage, and/or life remaining of consumables. This may be accomplished as part of the daily and/or weekly update of the data from the procedures run. A hub system may download any errors which occurred as part of the system and its instruments since the last data download.

The edge cloud may confirm and/or interrogate networked equipment and/or devices (e.g., on a predefined interval), e.g., to each of the local hub systems prior to use against the manufacturing acceptance test to monitor wear, degradation and/or life limited components. The check may be completed as part of a start-up or shutdown procedure of a local hub system. In the procedure, the data may be stored or sent to facility server or the edge cloud system and/or a manufacture to indicate when maintenance or service should be conducted. The check may be triggered based on a network congestion level (e.g., during times of no use or low use), local hub system down times, or scheduling related time (e.g., holidays, weekends, etc.)

Manufactures may do a type of acceptance check on the equipment and or device prior to packaging to confirm it meets acceptable performance. Using a system hub to check itself and/or other equipment and/or devices against the same metrics and/or acceptance testing prior to use could reassure and/or minimize issues when performing the procedure. The results may be evaluated against the initial acceptance check during manufacturing to confirm the shift in performance from storage condition and/or storage time and use. This may provide insight on when maintenance or service may be needed and/or indicate which components may be impacted based on performance indicators.

1. A computing system, the computing system comprising
a processor configured to:
receive, from at least one of a surgical hub or a data system on a local data network, a first collection of unredacted data associated with a first surgical procedure, wherein the first collection of unredacted data includes first patient personal data, first patient clinical data, and first other patient data;
receive, from the at least one of a surgical hub or a data system on the local data network, a second collection of respective unredacted data associated with a second surgical procedure;
train a machine learning (ML) model for optimizing clinical outcome and cost effectiveness using the first collection of unredacted data and the second collection of respective unredacted data;
generate first information that optimizes the clinical outcome and cost effectiveness of a third surgical procedure using the ML model; and
send the first information to a surgical hub from the at least one of a surgical hub or a data system.

2. The computing system of claim 1, wherein the processor is further configured to:
receive, from the surgical hub from the at least one of a surgical hub or a data system, a request for the first information that optimizes the clinical outcome and cost effectiveness of the third surgical procedure, wherein in response the processor is further configured to send the first information to the surgical hub.

3. The computing system of claim 1, wherein the computing system is coupled with a cloud computing system and the processor is further configured to:
redact the first collection of unredacted data; and
send the redacted first collection of unredacted data to the cloud computing system.

4. The computing system of claim 1, wherein the computing system is coupled with a cloud computing system and the processor further is configured to send to the cloud computing system the first information that optimizes the clinical outcome and cost effectiveness of the third surgical procedure.

5. The computing system of claim 1, wherein the first surgical procedure, the second surgical procedure, and the third surgical procedure are a same type of surgical procedure, wherein the first surgical procedure and the second surgical procedure are past surgical procedures, and wherein the third surgical procedure is a future surgical procedure.

6. The computing system of claim 1, wherein the first information that optimizes the clinical outcome and cost effectiveness of the third surgical procedure includes one or more of aspects of a surgical procedure plan associated with the third surgical procedure, such as a surgical choice, a surgical instrument selection, or a post-surgery care choice.

7. The computing system of claim 6, wherein the first information that optimizes the clinical outcome and cost effectiveness of the third surgical procedure further includes an operational parameter adjustment for a surgical instrument associated with the surgical instrument selection.

8. The computing system of claim 1, wherein the computing system is located on the local data network, and where the local data network is within a boundary protected by health insurance portability and accountability act (HIPAA) data rules.

9. The computing system of claim 1, wherein each of the first patient personal data, the first patient clinical data, and the first other patient data includes a patient identifier.

10. The computing system of claim 1, wherein the first patient personal data includes one or more of demographics information, such as age, gender, place of residence, occupation, or family status.

11. The computing system of claim 1, wherein the first patient clinical data includes one or more of pre-surgery data, in-surgery data, and post-surgery data.

12. The computing system of claim 1, wherein the first other patient data includes one or more of billing data associated with the first surgical procedure, payment data associated with the first surgical procedure, or reimbursement data associated with the first surgical procedure.

13. The computing system of claim 1, wherein second information that optimizes the clinical outcome and cost effectiveness of the third surgical procedure is received from a cloud computing system that is coupled with the computing system, and wherein the ML model is a part of the second information.

14. A computer-implemented method, the method comprising:
receiving, from at least one of a surgical hub or a data system on a local data network, a first collection of unredacted data associated with a first surgical procedure, wherein the first collection of unredacted data includes first patient personal data, first patient clinical data, and first other patient data;
receiving, from the at least one of a surgical hub or a data system on the local data network, a second collection of respective unredacted data associated with a second surgical procedure;
training a machine learning (ML) model for optimizing clinical outcome and cost effectiveness using the first collection of unredacted data and the second collection of respective unredacted data;
generating first information that optimizes the clinical outcome and cost effectiveness of a third surgical procedure using the ML model; and
sending the first information to a surgical hub from the at least one of a surgical hub or a data system.

15. The computer-implemented method of claim 14, further comprising:
receiving, from the surgical hub from the at least one of a surgical hub or a data system, a request for the first information that optimizes the clinical outcome and cost effectiveness of the third surgical procedure; and
in response, sending the first information to the surgical hub.

16. The computer-implemented method of claim 14, further comprising:
redacting the first collection of unredacted data; and
send the redacted first collection of unredacted data to a cloud computing system.

17. The computer-implemented method of claim 14, further comprising:
sending to a cloud computing system the first information that optimizes the clinical outcome and cost effectiveness of the third surgical procedure.

18. The computer-implemented method of claim 14, wherein the first surgical procedure, the second surgical procedure, and the third surgical procedure are a same type of surgical procedure, wherein the first surgical procedure and the second surgical procedure are past surgical procedures, and wherein the third surgical procedure is a future surgical procedure.

19. The computer-implemented method of claim 14, wherein the first information that optimizes the clinical outcome and cost effectiveness of the third surgical procedure includes one or more of aspects of a surgical procedure plan associated with the third surgical procedure, such as a surgical choice, a surgical instrument selection, or a post-surgery care choice.

20. The computer-implemented method of claim 14, where the local data network is within a boundary protected by health insurance portability and accountability act (HIPAA) data rules.
